(19) United States
(12) Patent Application Publication
(10) Pub. No.: US 2020/0337679 A1
(43) Pub. Date: Oct. 29, 2020

(54) ULTRASONIC SIGNAL PROCESSING APPARATUS, ULTRASONIC DIAGNOSTIC APPARATUS, ULTRASONIC SIGNAL PROCESSING METHOD, AND ULTRASONIC SIGNAL PROCESSING PROGRAM

(71) Applicant: Konica Minolta, Inc., Tokyo (JP)

(72) Inventors: Yasuhito Watanabe, Osaka (JP); Kazuya Takagi, Tokyo (JP)

(21) Appl. No.: 16/849,406

(22) Filed: Apr. 15, 2020

(30) Foreign Application Priority Data
Apr. 25, 2019 (JP) 2019-084167

(51) Int. Cl. A61B 8/08; A61B 8/00; G01N 29/04

(52) U.S. Cl. CPC A61B 8/485; A61B 8/4455; G01N 29/043; A61B 8/4488; A61B 8/085

(57) ABSTRACT

An ultrasonic signal processing apparatus includes: a push wave transmitter that causes the ultrasonic probe to transmit a push wave for causing displacement in a subject; a detection wave transmitter that causes the ultrasonic probe to transmit a detection wave after the transmission of the push wave; a detection wave receiver that receives an ultrasonic wave reflected from the region of the interest by using the ultrasonic probe and converts the ultrasonic wave into a reception signal; a phasing adder that sets a plurality of observation points in the region of the interest and performs phasing addition for each of the plurality of the observation points to generate an acoustic line signal; and a mechanical property calculator that calculates a mechanical property of the subject in the region of the interest based on an acoustic line signal for each of the plurality of the observation point.

The entire disclosure of Japanese patent Application No. 2019-084167, filed on Apr. 25, 2019, is incorporated herein by reference in its entirety.

### BACKGROUND

### Technological Field

The present disclosure relates to an ultrasonic diagnostic apparatus and an ultrasonic signal processing method, and more particularly to propagation speed analysis of a shear wave in a tissue and measurement of an elastic modulus of the tissue by using the shear wave.

### Description of the Related Art

An ultrasonic diagnostic apparatus is a medical examination apparatus that transmits ultrasonic waves from a plurality of transducers constituting an ultrasonic probe to the inside of a subject, receives ultrasonic reflected waves (echoes) caused by a difference in acoustic impedance of a tissue of the subject, and generates and displays an ultrasonic tomographic image showing a structure of an internal tissue of the subject based on an obtained electric signal.

In recent years, measurement of an elastic modulus of a tissue (shear wave speed measurement (SWSM), hereinafter, referred to as “ultrasonic measurement of an elastic modulus”), to which this technique of ultrasonography is applied, has been widely used for examination. This can non-invasively and easily measure the hardness of a tumor mass found in an organ or a body tissue, and is therefore useful in investigating the hardness of a tumor in cancer screening tests and assessing hepatic fibrosis in examination of liver disease.

In this ultrasonic measurement of the elastic modulus, a region of interest (ROI) in a subject is determined, and a push wave (a focused ultrasonic wave or an acoustic radiation force impulse (ARFI)), in which an ultrasonic wave is focused, is transmitted to a specific site in the subject from a plurality of transducers. Thereafter, transmission of an ultrasonic wave for detection (hereinafter, referred to as a “detection wave”) and reception of the reflected wave are repeated a plurality of times to conduct propagation analysis of a shear wave generated by acoustic radiation pressure of the push wave. Thus, the propagation speed of the shear wave, which represents the elastic modulus of a tissue, can be calculated (see, for example, JP 2016-97222 A).

In order to conduct propagation analysis of a shear wave, it is necessary to detect displacement at a plurality of positions in a subject. However, when a convex probe is used and an observation point, which is a target of displacement detection, is provided in the front direction of each element so as to improve the sensitivity of each element, the observation points are arranged on straight lines radiating from the probe. Therefore, there is a problem that the accuracy of the propagation speed of the shear wave decreases since the distance between the observation points in the propagation direction of the shear wave increases depending on the distance from the probe, and the spatial resolution decreases at a deeper portion as the distance from the probe increases.

### SUMMARY

The present disclosure has been made in light of the above problems, and an object thereof is to improve the reliability of measurement results of an elastic modulus when a convex probe is used in ultrasonic measurement of the elastic modulus.

To achieve the abovementioned object, according to an aspect of the present invention, there is provided an ultrasonic signal processing apparatus that excites a shear wave in a subject to analyze a propagation state of the shear wave by using a convex ultrasonic probe, and the ultrasonic signal processing apparatus reflecting one aspect of the present invention comprises: a push wave transmitter that causes the ultrasonic probe to transmit a push wave for causing displacement in a subject; a detection wave transmitter that causes the ultrasonic probe to transmit a detection wave after the transmission of the push wave, the detection wave passing through a region of interest which indicates an analysis target range in the subject; a detection wave receiver that receives an ultrasonic wave reflected from the region of the interest by using the ultrasonic probe and converts the ultrasonic wave into a reception signal, the ultrasound corresponding to the detection wave; a phasing adder that sets a plurality of observation points in the region of the interest and performs phasing addition for each of the plurality of the observation points to generate an acoustic line signal; and a mechanical property calculator that calculates a mechanical property of the subject in the region of the interest based on an acoustic line signal for each of the plurality of the observation points, wherein a distance between observation points along a propagation direction of a shear wave in the region of the interest is set to be not more than a distance between observation points along a propagation direction of a shear wave when a region closer to the ultrasonic probe than the region of the interest is set as the region of the interest.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a schematic diagram showing an overview of an SWS sequence including propagation analysis of a shear wave in an ultrasonic diagnostic apparatus according to an embodiment;

FIG. 2 is a functional block diagram of an ultrasonic diagnostic system including the ultrasonic diagnostic apparatus;

FIG. 3A is a schematic view showing a position of a transmission focal point F of a push wave generated by a push wave generator;

FIG. 3B is a schematic view showing a configuration overview of a detection wave pulse generated by a detection wave generator;

FIG. 4A is a functional block diagram showing a configuration of a transmission beam former;

FIG. 4B is a functional block diagram showing a configuration of a reception beam former;

FIG. 5A is a schematic view showing an overview of detection wave transmission;

FIG. 5B is a schematic view showing an overview of reflected detection wave reception;

FIG. 6A is a schematic view showing an overview of a method of calculating a propagation path of an ultrasonic wave in a delay processor;

FIG. 6B is a schematic view showing an overview of a propagation analysis in a speed calculator;

FIG. 7 is a flowchart showing the operation of SWSM processing in the ultrasonic diagnostic apparatus;

FIG. 8A is a schematic view showing an overview of ultrasonic wave transmission for B-mode image generation;

FIG. 8B is a schematic view showing an overview of reflected ultrasonic wave reception for the B-mode image generation;

FIG. 9 is a schematic view showing an overview of a method of calculating a propagation path of an ultrasonic wave for the B-mode image generation;

FIG. 10A is a schematic diagram showing a relationship between a measurable range and a region of interest;

FIG. 10B is a schematic view showing an overview of detection wave transmission;

FIG. 11A is a schematic diagram showing a relationship between observation points and regions of interest by a similar method for the B-mode image generation;

FIG. 11B is a schematic diagram showing a relationship with respect to regions of interest according to an embodiment; and

FIG. 12 is a flowchart showing the operation of SWSM processing according to Modification 3.

### DETAILED DESCRIPTION OF EMBODIMENTS

Hereinafter, one or more embodiments of the present invention will be described with reference to the drawings. However, the scope of the invention is not limited to the disclosed embodiments.

<<Development of Mode for Carrying Out Invention>>

The inventor(s) have conducted various studies to prevent the measurement accuracy from decreasing depending on the depth of a region of interest in ultrasonic measurement of elasticity using a convex probe.

As described above, in the ultrasonic measurement of elasticity, a shear wave is excited in a subject by a push wave, and an elastic modulus is measured by measuring a propagation state of the shear wave. This is because the elastic modulus (Young's modulus) of a tissue is substantially proportional to the square of the propagation speed of the shear wave. Therefore, in the ultrasonic measurement of elasticity, displacement in the subject is detected by repeating transmission and reception of a detection wave after the transmission of the push wave, and a position of the wavefront of the shear wave is estimated by analyzing the time-series change of the displacement. Then, the moving speed of the wavefront is calculated as the moving speed of the shear wave. For the positional estimation of the wavefront of the shear wave, there is a method in which a plurality of observation points are provided in the subject, the time at which the displacement amount becomes maximum (peak) at each observation point (hereinafter, referred to as the “peak time”) is detected, and the wavefront of the shear wave is regarded to have passed through the observation points at the peak times.

The speed of the shear wave is calculated by dividing the distance between the observation points by the difference between the peak times. Therefore, as the distance between the observation points increases, the propagation speed of the shear wave is spatially averaged, and the distance resolution decreases. In addition, the detection accuracy of the displacements at the observation points depends not only on the magnitudes of the displacement at the observation points and the signal to noise ratio (SNR) of the reflected detection waves from the observation points, but also on the intensity (amplitude) of the reflected detection waves. Therefore, if the SNR of the reflected ultrasonic waves from the observation points is low for some reason or if the detection wave reflectance at the observation points is low and the reflected ultrasonic waves are weak, there may be a case where the detection accuracy of the displacement decreases, and the reliability of the propagation speed of the shear wave decreases. In particular, in so-called point-type measurement, in which a region of interest is narrowed and the average of the propagation speeds for the entire region of interest is calculated in order to improve the accuracy of the propagation speed of the shear wave, if the number of observation points that can be used for the speed analysis of the shear wave is insufficient, there arises a problem that the reliability of the propagation speed of the shear wave decreases or that the speed analysis of the shear wave cannot be conducted.

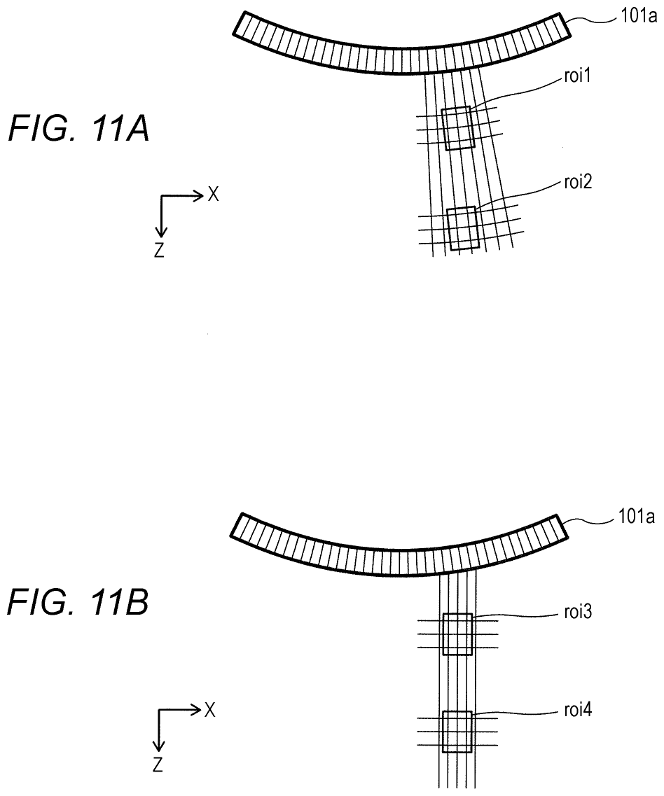

Meanwhile, when a convex probe is used, observation points are generally provided in the front direction of each transducer as shown in FIG. 11A. That is, the observation points are provided on straight lines radiating from the center point of a circular arc constituting the surface of the convex probe. The reason is that, as described above, the transducer has the highest sensitivity in the front direction thereof. Thus, this is an effective technique of improving the SNR of the acoustic line signals. However, since the distance between the observation points in the x direction, which is the propagation direction of the shear wave, increases with depth, the distance between the observation points in the x direction is different due to the depth for two regions of interest roi 1 and roi 2 with the same area as shown in FIG. 11A. More specifically, the distance between the observation points in the x direction for the region of interest roi 2 is longer than that for the region of interest roi 1, and the number of observation points is less for the region of interest roi 2 than that for the region of interest roi 1. Accordingly, if the propagation speed of the shear wave is averaged in the propagation direction and the distance resolution is decreased as well as the signal quality (amplitude and SNR) of the acoustic line signals is low, the propagation analysis of the shear wave possibly becomes difficult due to the insufficient number of observation points. Therefore, the inventor(s) have studied a method of transmitting and receiving a detection wave and a method of setting an observation point when a convex probe is used, and have arrived at an ultrasonic signal processing apparatus, an ultrasonic diagnostic apparatus and an ultrasonic signal processing method according to the present disclosure.

Hereinafter, an ultrasonic image processing method according to an embodiment and an ultrasonic diagnostic apparatus using the same will be described in detail with reference to the drawings.

### EMBODIMENTS

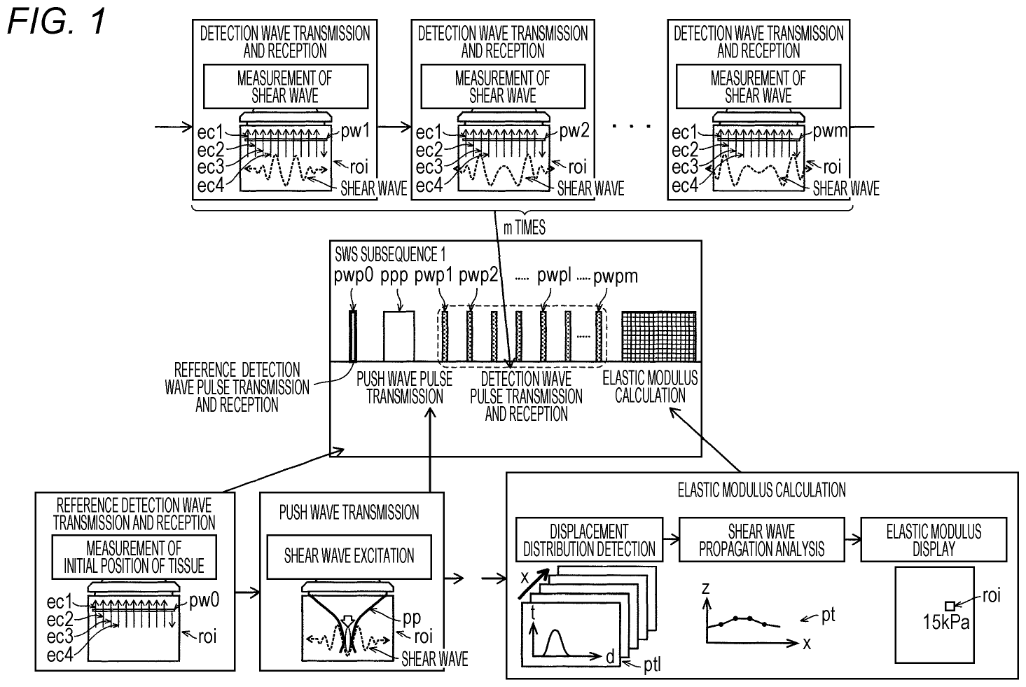

An ultrasonic diagnostic apparatus 100 performs processing of calculating a propagation speed of a shear wave, which represents an elastic modulus of a tissue, by an ultrasonic measurement method of an elastic modulus. FIG. 1 is a schematic diagram showing an overview of an SWS sequence by the ultrasonic measurement method of the elastic modulus in the ultrasonic diagnostic apparatus 100. As shown in the middle frame of FIG. 1, the processing of the ultrasonic diagnostic apparatus 100 includes the steps of “reference detection wave pulse transmission and reception,” “push wave pulse transmission,” “detection wave pulse transmission and reception,” and “elastic modulus calculation.”

In the step of the “reference detection wave pulse transmission and reception,” a reference detection wave pulse pwp0 is transmitted to an ultrasonic probe to cause a plurality of transducers to transmit a detection wave pw0 and receive a reflected wave ec in a range corresponding to a region of interest roi in a subject, thereby generating an acoustic line signal, which is reference of the initial position of the tissue.

In the step of the “push wave pulse transmission,” a push wave pulse ppp is transmitted to the ultrasonic probe to cause the plurality of transducers to transmit a push wave pp, which is obtained by converging ultrasonic waves, to a specific site in the subject, thereby exciting a shear wave passing through the region of interest roi.

Then, in the step of the “detection wave pulse transmission and reception,” a detection wave pulse pwp1 is transmitted to the ultrasonic probe to cause the plurality of transducers to transmit a detection wave pw1 and receive the reflected wave ec a plurality of times, thereby measuring the propagation state of the shear wave in the region of interest roi. In the step of the “elastic modulus calculation,” displacement distribution pt1 of a tissue, which is associated with the propagation of the shear wave, is calculated first in time series. Next, the propagation analysis of the shear wave is conducted to calculate the propagation speed of the shear wave, which represents the elastic modulus of the tissue, from time series changes of the displacement distribution pt1. At the end, the elastic modulus is displayed.

The series of steps associated with one-time shear wave excitation based on the transmission of the push wave pp described above is called the “shear wave speed (SWS) sequence.”

<Ultrasonic Diagnostic System 1000>

1. Overview of Apparatus

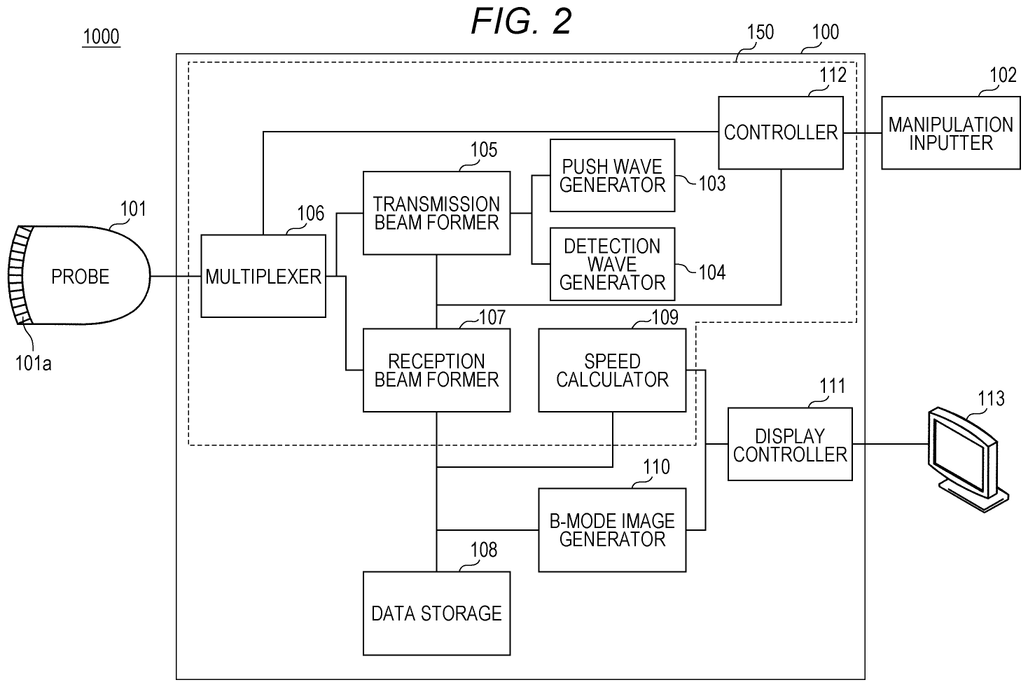

An ultrasonic diagnostic system 1000 including the ultrasonic diagnostic apparatus 100 according to an embodiment will be described with reference to the drawings. FIG. 2 is a functional block diagram of the ultrasonic diagnostic system 1000 according to an embodiment. As shown in FIG. 2, the ultrasonic diagnostic system 1000 has: an ultrasonic probe 101 (hereinafter, referred to as a “probe 101”) in which a plurality of transducers (transducer array) 101a that transmit ultrasonic waves toward a subject and receive the reflected waves are arrayed on the front end surface; the ultrasonic diagnostic apparatus 100 that causes the probe 101 to transmit and receive ultrasonic waves and generates an ultrasonic signal based on an output signal from the probe 101; a manipulation inputter 102 that accepts manipulation input from an examiner; and a display 113 that displays an ultrasonic image on a screen. The probe 101, the manipulation inputter 102 and the display 113 are each configured to be connectable to the ultrasonic diagnostic apparatus 100.

Next, each element externally connected to the ultrasonic diagnostic apparatus 100 will be described.

2. Probe 101

The probe 101 is a so-called convex probe having the transducer array (101a) including the plurality of transducers 101a aligned in an arc. The probe 101 converts a pulsed electric signal (hereinafter, referred to as a “transmission signal”), which is supplied from a transmission beam former 105 described later, into a pulsed ultrasonic wave. In a state where a transducer surface of the probe 101 is in contact with a surface of a subject via an ultrasonic gel or the like, the probe 101 transmits an ultrasonic beam composed of a plurality of ultrasonic waves emitted from the plurality of transducers 101a toward a measurement target. Then, the probe 101 receives a plurality of reflected detection waves (hereinafter, referred to as the “reflected waves”) from the subject, converts the reflected waves into the respective electric signals by the plurality of transducers 101a, and supplies the electric signals to the ultrasonic diagnostic apparatus 100.

3. Manipulation Inputter 102

The manipulation inputter 102 accepts various manipulation input such as various settings and manipulations for the ultrasonic diagnostic apparatus 100 from an examiner and outputs the inputter to a controller 112 of the ultrasonic diagnostic apparatus 100.

The manipulation inputter 102 may be, for example, a touch panel integrated with the display 113. In this case, various settings and manipulations of the ultrasonic diagnostic apparatus 100 can be performed through touch manipulation and drag manipulation on operation keys displayed on the display 113, and the ultrasonic diagnostic apparatus 100 is configured to be manipulatable via the touch panel. Alternatively, the manipulation inputter 102 may be, for example, a keyboard with keys for various manipulations, buttons for various manipulations, a manipulation panel with a lever and the like, a mouse or the like.

<Overview of Configuration of Ultrasonic Diagnostic Apparatus 100>

Next, an ultrasonic diagnostic apparatus 100 according to Embodiment 1 will be described.

The ultrasonic diagnostic apparatus 100 has: a multiplexer 106 that selects each transducer to be used for transmission or reception from among a plurality of transducers 101a of a probe 101 and secures input and output with respect to the selected transducers; a transmission beam former 105 that controls timing of applying a high voltage to each of the transducers 101a of the probe 101 for ultrasonic wave transmission; and a reception beam former 107 that performs reception beamforming based on the reflected waves received by the probe 101 to generate an acoustic line signal.

Moreover, the ultrasonic diagnostic apparatus 100 has: a push wave generator 103 that transmits a push wave pulse ppp to the plurality of transducers 101a; and a detection wave generator 104 that transmits a detection wave pulse pwp1 a plurality (m) of times to the plurality of transducers 101a after the push wave pulse ppp.

Furthermore, the ultrasonic diagnostic apparatus 100 includes: a data storage 108 that stores the acoustic line signal outputted by the reception beam former 107; a speed calculator 109 that performs propagation analysis of the shear wave in a region of interest roi based on the acoustic line signal; a B-mode image generator 110 that generates a B-mode image from the acoustic line signal; a display controller 111 that forms a display image from at least one of the B-mode image or the result of the propagation analysis and causes the display 113 to display the display image; and the controller 112 that sets the region of interest roi, which represents an analysis target range in a subject, based on the manipulation input from the manipulation inputter 102 as well as controls each constituent.

Of these elements, the multiplexer 106, the transmission beam former 105, the reception beam former 107, the push wave generator 103, the detection wave generator 104, the speed calculator 109 and the controller 112 constitute an ultrasonic signal processing circuit 150.

Each element constituting the ultrasonic signal processing circuit 150, the B-mode image generator 110 and the display controller 111 can be each realized by, for example, a hardware circuit such as a field programmable gate array (FPGA) or an application specific integrated circuit (ASIC). Alternatively, the configurations may be realized by processors such as a central processing unit (CPU) and a graphics processing unit (GPU) and software. The configuration using the GPU in particular is called a general-purpose computing on graphics processing unit (GPGPU). These constituents can each be a single circuit component or an aggregate of a plurality of circuit components. Alternatively, a plurality of constituents can be combined into a single circuit component or can be an aggregate of a plurality of circuit components.

The data storage 108 is a computer-readable recording medium, and, for example, a flexible disk, a hard disk, an MO, a DVD, a BD, a semiconductor memory or the like can be used. Moreover, the data storage 108 may be a storage apparatus connected to the ultrasonic diagnostic apparatus 100.

Note that the ultrasonic diagnostic apparatus 100 according to the embodiment is not limited to an ultrasonic diagnostic apparatus with the configuration shown in FIG. 2. For example, the configuration may not need the multiplexer 106, or the configuration may be such that the transmission beam former 105 and the reception beam former 107 or parts thereof are built in the probe 101.

<Configuration of Each Constituent of Ultrasonic Diagnostic Apparatus 100>

Next, the configuration of each block included in the ultrasonic diagnostic apparatus 100 will be described.

1. Controller 112

Generally, in a state where a B-mode image, which is a tomographic image of a subject acquired in real time by the probe 101, is displayed on the display 113, a manipulator designates an analysis target position in the subject with the B-mode image displayed on the display 113 as an index and inputs the analysis target position into the manipulation inputter 102. The controller 112 sets a region of interest roi, which is an analysis target range, with the information designated by the manipulator from the manipulation inputter 102 as input. Herein, since one value is acquired for the mechanical properties of the subject in the entire region of interest roi, the region of interest roi preferably has a narrow range that does not include inside thereof a plurality of target positions at which the mechanical properties are acquired. Alternatively, the controller 112 may set the region of interest roi with, as reference, the position of the transducer array (101a) including the plurality of transducers 101a in the probe 101. For example, the region of interest roi may be set in the front direction of a transducer 101a slightly away from the center of the transducer array (101a) including the plurality of transducers 101a.

Moreover, the controller 112 controls other blocks of the ultrasonic diagnostic apparatus 100 described later based on an instruction from the manipulation inputter 102.

2. Push Wave Generator 103

The push wave generator 103 acquires information indicating the region of interest roi from the controller 112 and sets a specific point in the vicinity of the region of interest roi. Then, the transmission of the push wave pulse ppp from the transmission beam former 105 to the plurality of transducers 101a causes the plurality of transducers 101a to transmit a push wave pp, in which an ultrasonic beam is focused, to a specific site in the subject corresponding to the specific point (hereinafter, referred to as a “transmission focal point FP”). Accordingly, a shear wave is excited at the specific site in the subject.

Specifically, based on the information indicating the region of interest roi, the push wave generator 103 decides, as shown below, a position of the transmission focal point FP of the push wave and a transducer array that transmits the push wave ppp (hereinafter, referred to as a “push wave transmission transducer array Px).

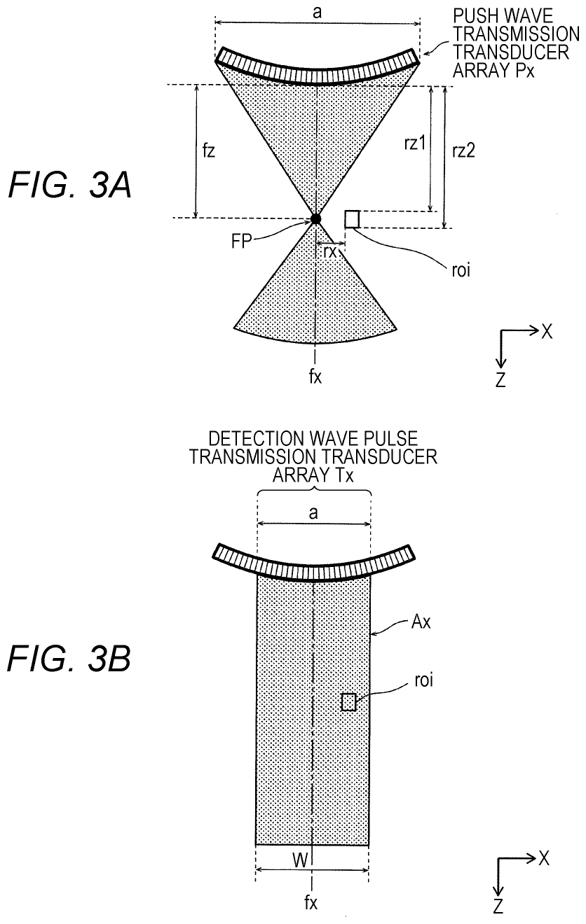

FIG. 3A is a schematic view showing the position of the transmission focal point FP of the push wave ppp generated by the push wave generator 103. In the present embodiment, as shown in FIG. 3A, an array direction transmission focal position fx of the transmission focal point FP exists in the front direction of the transducer at the array direction center position of the transducer array 101a. Herein, the array direction transmission focal position fx of the transmission focal point FP and the region of interest roi are separated by an array direction distance rₓ. Moreover, a depth direction transmission focal position fz has a value between the minimum depth r_{z1} and the maximum depth r_{z2} of the region of interest roi.

Furthermore, the push wave transmission transducer array Px is set based on the depth direction transmission focal position fz. In the present embodiment, the length of the push wave pulse transmission transducer array Px is a length a of part of the array of the plurality of transducers 101a.

The information indicating the position of the transmission focal point FP and the push wave transmission transducer array Px is outputted to the transmission beam former 105 together with a pulse width PW and an application start time PT of the push pulse ppp as transmission control signals. In addition, a time interval PI of the application start time PT may be included. Note that the pulse width PW, the application start time PT and the time interval PI of the push wave pulse ppp will be described later.

Note that the positional relationship between the region of interest roi and the transmission focal point FP is not limited to the above and may be changed as appropriate depending on the form of the site of the subject to be examined or the like.

Note that “focusing” the ultrasonic beam according to the push wave refers to focusing the ultrasonic beam into a focused beam, that is, reducing the area irradiated with the ultrasonic beam after the transmission and reaching the minimum value at a specific depth, and is not limited to a case where the ultrasonic beam is focused on one point. In this case, the “transmission focal point FP” refers to the array direction center of the ultrasonic beam at the depth where the ultrasonic beam is focused.

3. Detection Wave Generator 104

The detection wave generator 104 inputs the information indicating the region of interest roi from the controller 112 and causes a plurality of transducer 101a belonging to a detection wave pulse transmission transducer array Tx to transmit a detection wave pw such that the ultrasonic beam passes through the region of interest roi by transmitting a detection wave pulse pwp1 from the transmission beam former 105 to the plurality of transducers 101a a plurality of times. Specifically, based on the information indicating the region of interest roi, the detection wave generator 104 decides a transducer array to which the detection wave pulse pwp1 is transmitted (hereinafter, referred to as a “detection wave pulse transmission transducer array Tx”) such that the ultrasonic beam passes through the region of interest roi. At this time, the number of transmission times (m) of the detection wave pulse pwp1 may be, for example, 30 to 100 times. And, the transmission interval of the detection wave pulse pwp1 may be, for example, 100 μsec to 150 μsec. However, it is needless to say that these application conditions are not limited to the above and can be changed as appropriate.

FIG. 3B is a schematic view showing an overview of a configuration of the detection wave pulse pwp1 generated by the detection wave generator 104. As shown in FIG. 3B, the detection wave generator 104 sets the detection wave pulse transmission transducer array Tx such that the detection wave that is a plane wave passes through the entire region of interest roi. The length a of the detection wave pulse transmission transducer array Tx is preferably set to be equal to or greater than a detection wave reception region width W including the region of interest roi, with the array direction transmission focal position fx of the transmission focal point FP as the array direction center. In this example, both ends of the detection wave reception region width W is set at the ends of the detection wave pulse transmission transducer array Tx in the array direction. Since the detection wave pw is a plane wave, the detection wave pw propagates in the z direction, which is the depth direction. Therefore, the region of interest roi is included in an ultrasonic irradiation region Ax with a margin by a distance β at both ends in the x direction. Moreover, the configuration of the detection wave pulse transmission transducer array Tx may be such that the absolute value of an angle φ between the front direction of the transducers 101a at both ends of the array Tx and the z direction is equal to or less than a predetermined maximum value φₘₐₓ. Note that the detection wave is not limited to a plane wave, and the transmission wave only needs to pass through the region of interest roi. The detection wave may be an unfocused wave other than the plane wave or may be a focused wave which is focused at a sufficiently deep position with respect to the depth of the region of interest roi (e.g., three times the depth of the region of interest roi).

The information indicating the detection wave pulse transmission transducer array Tx is outputted to the transmission beam former 105 together with the pulse width of the detection wave pulse pwp1 as transmission control signals.

4. Transmission Beam Former 105

The transmission beam former 105 is a circuit that is connected to the probe 101 via the multiplexer 106 and, to transmit the ultrasonic from the probe 101, controls the timing of applying a high voltage to each of the plurality of transducers included in the push wave transmission transducer array Px or the detection wave pulse transmission transducer array Tx, which correspond to all or some of the plurality of transducers 101a present in the probe 101.

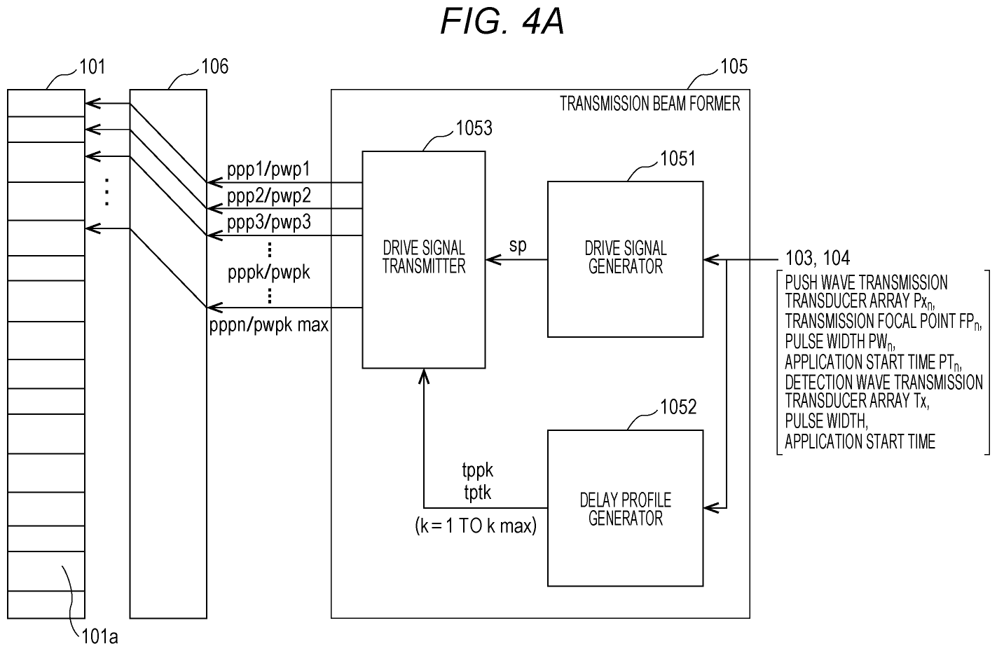

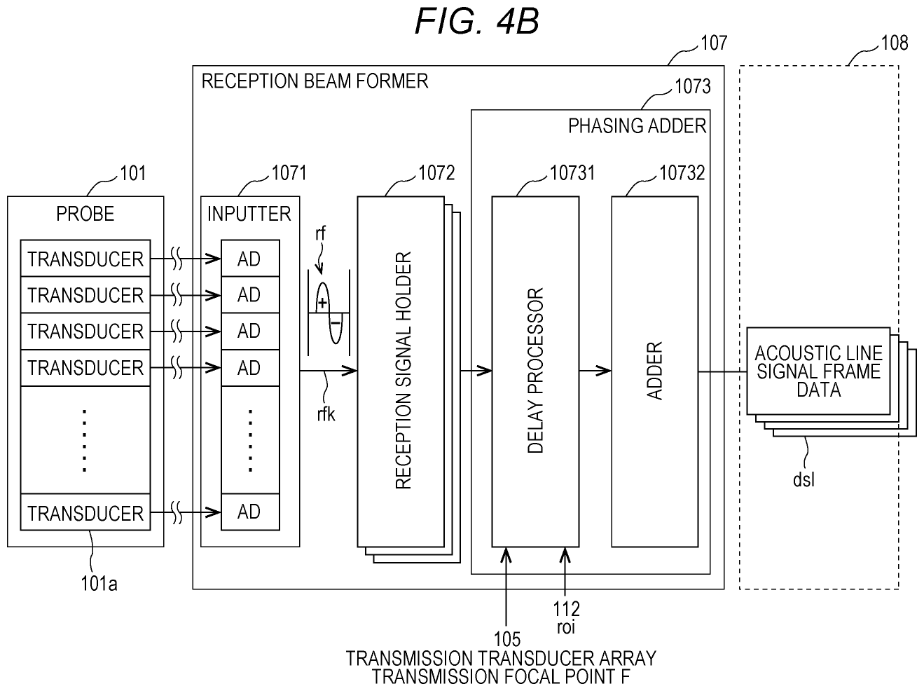

FIG. 4A is a functional block diagram showing a configuration of the transmission beam former 105. As shown in FIG. 4A, the transmission beam former 105 includes a drive signal generator 1051, a delay profile generator 1052 and a drive signal transmitter 1053.

(1) Drive Signal Generator 1051

The drive signal generator 1051 is a circuit that generates a pulse signal sp for causing a transmission transducer, which corresponds to some or all of the transducers 101a present in the probe 101, to transmit an ultrasonic beam based on the information indicating the push wave transmission transducer array Px or the detection wave pulse transmission transducer array Tx, the information indicating the pulse width PW and the application start time PT of the push wave pulse ppp, and the information indicating the pulse width and the application start time of the detection wave pulse pwp1 among the transmission control signals from the push wave generator 103 or the detection wave generator 104.

(2) Delay Profile Generator 1052

The delay profile generator 1052 is a circuit that sets and outputs, for each transducer, a delay time tppk (k is a natural number from one to the number of transducers 101a kmax) from the application start time PT, which decides transmission timing of an ultrasonic beam, based on the information indicating the push wave transmission transducer array Px and the position of the transmission focal point FP among the transmission control signals obtained from the push wave generator 103. Moreover, the delay profile generator 1052 sets and outputs, for each transducer, a delay time tptk (k is a natural number from one to the number of transducers 101a kmax) from the application start time PT, which decides the transmission timing of the ultrasonic beam, based on the information indicating the detection wave pulse transmission transducer array Tx among the transmission control signals obtained from the detection wave generator 104. Accordingly, the transmission of the ultrasonic beam is delayed for each transducer by the delay time, and the ultrasonic beam is focused.

(3) Drive Signal Transmitter 1053

The drive signal transmitter 1053 performs push wave transmission processing of supplying the push wave pulse ppp for causing each transducer included in the push wave transmission transducer array Px among the plurality of transducers 101a present in the probe 101 to transmit a push wave based on the pulse signal sp from the drive signal generator 1051 and the delay time tppk from the delay profile generator 1052. The push wave transmission transducer array Px is selected by the multiplexer 106.

A push wave that produces physical displacement in a living body requires much greater power than a transmission pulse used for normal B-mode display or the like. That is, as a drive voltage to be applied to a pulser (ultrasonic wave generator), generally even 30 to 40 V can be acceptable for acquisition of a B-mode image, whereas a push wave requires, for example, 50 V or more. In addition, the transmission pulse length is about several μsec for the acquisition of the B-mode image, whereas the push wave requires a transmission pulse length of several hundreds of μsec per transmission in some cases.

In the present embodiment, the push wave pulse ppp is transmitted to the plurality of transducers 101a from the drive signal transmitter 1053 at the application start time PT. The push wave pulse ppp is a burst signal having a predetermined pulse width PW (time length), a predetermined voltage amplitude (+V to −V) and a predetermined frequency. Specifically, the pulse width PW may be, for example, 100 to 200 μsec, the frequency may be, for example, 6 MHz, and the voltage amplitude may be, for example, be +50 V to −50 V. However, it is needless to say that the application conditions are not limited to the above.

In addition, the drive signal transmitter 1053 performs detection wave transmission processing of supplying the detection wave pulse pwp1 for causing each transducer included in the detection wave pulse transmission transducer array Tx among the plurality of transducers 101a present in the probe 101 to transmit an ultrasonic beam. The detection wave pulse transmission transducer array Tx is selected by the multiplexer 106. However, the configuration related to the supply of the detection wave pulse pwp1 is not limited to the above, and, for example, may not use the multiplexer 106.

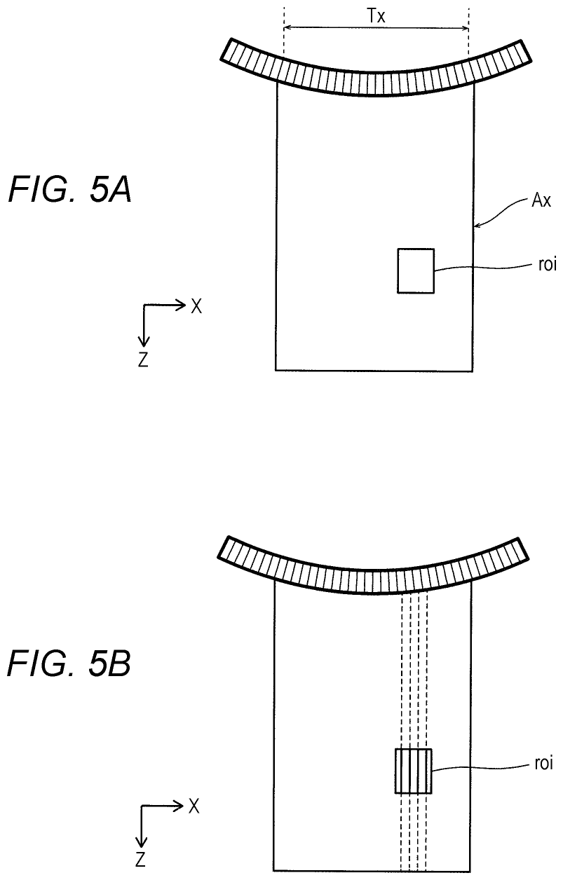

FIG. 5A is a schematic view showing an overview of detection wave transmission. The delay time tptk is applied to the transducers included in the detection wave pulse transmission transducer array Tx, and the detection wave pw is transmitted from the detection wave pulse transmission transducer array Tx. Accordingly, as shown in FIG. 5A, a plane wave that travels in the depth direction (z direction) of the subject is transmitted from each transducer in the detection wave pulse transmission transducer array Tx. A region in a plane, which corresponds to the range in the subject to which the detection wave reaches and includes the detection wave pulse transmission transducer array Tx, is a detection wave irradiation region Ax.

After the transmission of the push wave pulse ppp, the transmission beam former 105 transmits the detection wave pulse pwp1 a plurality of times based on the transmission control signals from the detection wave generator 104. Each time of a series of detection wave pulse pwp1 transmission performed a plurality of times to the same detection wave pulse transmission transducer array Tx after one push wave pulse ppp transmission is referred to as a “transmission event.”

5. Reception Beam Former 107

The reception beam former 107 is a circuit that generates acoustic line signals for a plurality of observation points Pij present in both the detection wave irradiation region Ax and in the region of interest roi to generate a sequence of acoustic line signal frame data ds1 (1 is a natural number from one to m, referred to as acoustic line signal frame data ds1 in a case where the number is not distinguished) based on the reflected waves from the tissue of the subject received in time series by the plurality of transducers 101a in response to the respective detection wave pulses pwp1 of a plurality of times. That is, after the transmission of the detection wave pulse pwp1, the reception beam former 107 generates acoustic line signals from the electric signals obtained by the plurality of transducers 101a based on the reflected waves received by the probe 101. Herein, in the region of interest roi, i is a natural number indicating the coordinate in the x direction, and j is a natural number indicating the coordinate in the z direction. Note that an “acoustic line signal” is a signal obtained by phasing addition processing on a reception signal (RF signal).

FIG. 4B is a functional block diagram showing a configuration of the reception beam former 107. The reception beam former 107 includes an inputter 1071, a reception signal holder 1072 and a phasing adder 1073.

(1) Inputter 1071

The inputter 1071 is a circuit that is connected to the probe 101 via the multiplexer 106 and generates a reception signal (RF signal) based on the reflected wave at the probe 101. Herein, a reception signal rfk (k is a natural number from one to n) is a so-called RF signal, which is obtained by subjecting the electric signal obtained by conversion of the reflected wave received by each transducer based on the transmission of the detection wave pulse pwp1 to A/D conversion. The reception signal rfk is composed of a string of signals (reception signal string) that is continuous in the transmission direction (the depth direction of the subject) of ultrasonic wave received by each reception transducer rwk.

The inputter 1071 generates a string of the reception signals rfk for each reception transducer rwk at each transmission event based on the reflected waves obtained by the respective reception transducers rwk. The reception transducer array is constituted by a transducer array corresponding to some or all of the plurality of transducers 101a present in the probe 101 and is selected by the multiplexer 106 based on an instruction from the controller 112. In this example, all of the plurality of transducers 101a are selected as the reception transducer array. Accordingly, as shown in FIG. 5B showing an overview of the reflected detection wave reception, the reflected waves from the observation points present in the entire detection wave irradiation region Ax can be received by one reception processing using all the transducers to generate the reception signal strings for all transducers. The generated reception signals rfk are outputted to the reception signal holder 1072.

(2) Reception Signal Holder 1072

The reception signal holder 1072 is a computer-readable recording medium, and, for example, a semiconductor memory or the like can be used. The reception signal holder 1072 inputs the reception signal rfk for each reception transducer rwk from the inputter 1071 in synchronization with the transmission event and holds the reception signals rfk until one acoustic line signal frame data is generated.

Note that the reception signal holder 1072 may be part of the data storage 108.

(3) Phasing Adder 1073

The phasing adder 1073 is a circuit that generates an acoustic line signal ds by performing addition for all reception transducers Rpk after delay processing is performed on the reception signals rfk received by the reception transducers Rpk included in a detection wave pulse reception transducer array Rx from the observation points Pij in the region of interest roi in synchronization with the transmission event. Herein, the observation points Pij are arranged such that the interval in the array direction (x direction) does not depend on the position of the region of interest roi in the depth direction (z direction). That is, in two regions of interest roi, which are at the same position in the array direction (x direction) and different in the depth direction (z direction), the intervals between the observation points Pij in the array direction (x direction) are the same. Specifically, the observation points Pij are arranged at regular intervals in the z direction on straight lines which extend in the depth direction (z direction) and are parallel to each other. Note that each of the straight lines, which extend in the depth direction (z direction) and are parallel to each other, may be a straight line passing through the center of any of the reception transducers Rpk. Accordingly, as shown in FIG. 11B, the intervals between the observation points Pij in the x direction are the same for both a shallow region of interest roi 3 and a deep region of interest roi 4, and the numbers of observation points Pij included in the regions of interest are equal if the areas of the regions of interest roi are the same. Note that the observation points Pij may be provided one by one on straight lines which extend in the depth direction (z direction) and are parallel to each other. In this case, the positions of the plurality of observation points Pij are preferably the same in the z direction. The detection wave pulse reception transducer array Rx is constituted by the reception transducers Rpk corresponding to some or all of the plurality of transducers 101a present in the probe 101 and is selected by the phasing adder 1073 and the multiplexer 106 based on an instruction from the controller 112. In this example, a transducer array, which includes at least all the transducers constituting the detection wave pulse transmission transducer array Tx for each transmission event, is selected as the reflected wave pulse reception transducer array Rx.

The phasing adder 1073 includes a delay processor 10731 and an adder 10732 for performing processing on the reception signals rfk.

a) Delay Processor 10731

The delay processor 10731 is a circuit that compensates reception signals rfk for the reception transducers Rpk in the detection wave pulse reception transducer array Rx according to an arrival time difference (delay amount) of the reflected ultrasonic wave to each reception transducer Rpk, which is obtained by dividing a difference in distance between the observation point Pij and the reception transducer Rpk by a sound speed value, and identifies the reception signal as a received signal for the reception transducer Rpk based on the reflected ultrasonic wave from the observation point Pij.

Calculation of Transmission Time

The delay processor 10731 specifies the transmission path to the observation point Pij for the transmission event and calculates the transmission time by dividing the distance by the sound speed. The transmission path can be, for example, a straight path from the center of the detection wave pulse transmission transducer array Tx to the observation point Pij. Note that the transmission path is not limited to this and may be, for example, the shortest path from the center of the detection wave pulse transmission transducer array Tx to an arbitrary point having the same depth as the observation point Pij.

Calculation of Reception Time

In response to the transmission event, the delay processor 10731 specifies, for the observation point Pij, a reception path for arrival to the reception transducer included in the detection wave reception transducer array from the reflection at the observation point Pij and calculates the reception time by dividing the distance by the sound speed. The reception path may be, for example, a straight path from the observation point Pij to the reception transducer.

Calculation of Delay Amount

Next, the delay processor 10731 calculates the total propagation time to each reception transducer from the transmission time and the reception time and calculates a delay amount, which applies to the reception signal string rfk for each reception transducer based on the total propagation time.

Delay Processing

Next, the delay processor 10731 identifies, as a signal for the reception transducer based on the reflected wave from the observation point Pij, a reception signal rfk equivalent to the delay amount (a reception signal corresponding to the time obtained by subtracting the delay amount) from the reception signal string rfk for each reception transducer.

In response to the transmission event, the delay processor 10731 inputs the reception signal rfk from the reception signal holder 1072 and identifies the reception signal rfk to each reception transducer Rpk for all the observation points Pij positioned in the region of interest roi.

b) Adder 10732

The adder 10732 is a circuit that inputs the reception signals rfk that are identified for the reception transducer Rpk and outputted from the delay processor 10731, adds the reception signals rfk, and generates an acoustic line signal dsij obtained by phasing addition for the observation point Pij.

In addition, the acoustic line signal dsij for the observation point Pij may be generated by performing addition after multiplying the reception signal rfk identified for each reception transducer Rpk by a reception apodization (weight sequence). The reception apodization is a sequence of weight coefficients applied to the received signal to the reception transducer Rpk in the detection wave pulse transmission transducer array Rx. For example, the reception apodization is set so that the weight of the transducer positioned at the center of the detection wave pulse transmission transducer array Rx becomes maximum, the central axis of the reception apodization distribution coincides with the central axis Rxo of the detection wave pulse transmission transducer array, and the distribution has a shape symmetric about the central axis. The shape of the distribution is not particularly limited. Note that the reception apodization is not limited to the above-described case and, for example, may be set so that the weight of the transducer positioned at the center of the transmission transducer array Tx in the array direction becomes maximum.

The adder 10732 generates acoustic line signals dsij for all the observation points Pij present in the region of interest roi to generate the acoustic line signal frame data ds1.

Then, the transmission and reception of the detection wave pulse pwp1 are repeated in synchronization with the transmission event, and the acoustic line signal frame data ds1 for all the transmission events are generated. The generated acoustic line signal frame data ds1 is outputted to and stored in the data storage 108 for each transmission event.

6. Speed Calculator 109

The speed calculator 109 is a circuit that detects the displacement of the tissue in the region of interest roi from the sequence of the acoustic line signal frame data ds1 and calculates the speed of the shear wave.

The speed calculator 109 acquires one frame of the acoustic line signal frame data ds1 included in the sequence of the acoustic line signal frame data ds1 and the acoustic line signal frame data (reference acoustic line signal frame data) ds0 serving as reference. The reference acoustic line signal frame data ds0 is a reference signal for extracting displacement caused by a shear wave in the acoustic line signal frame data ds1 for each transmission event. Specifically, the reference acoustic line signal frame data ds0 is frame data of acoustic line signals acquired from the region of interest roi before the transmission of the push wave pulse ppp. Then, the speed calculator 109 detects the displacement at each observation point Pij from the difference between the acoustic line signal frame data ds1 and the reference acoustic line signal frame data ds0. Subsequently, by repeating this processing, the speed calculator 109 detects a time-series change of the displacement at each of the observation points Pij and detects a peak time Tij of the displacement at the observation point Pij.

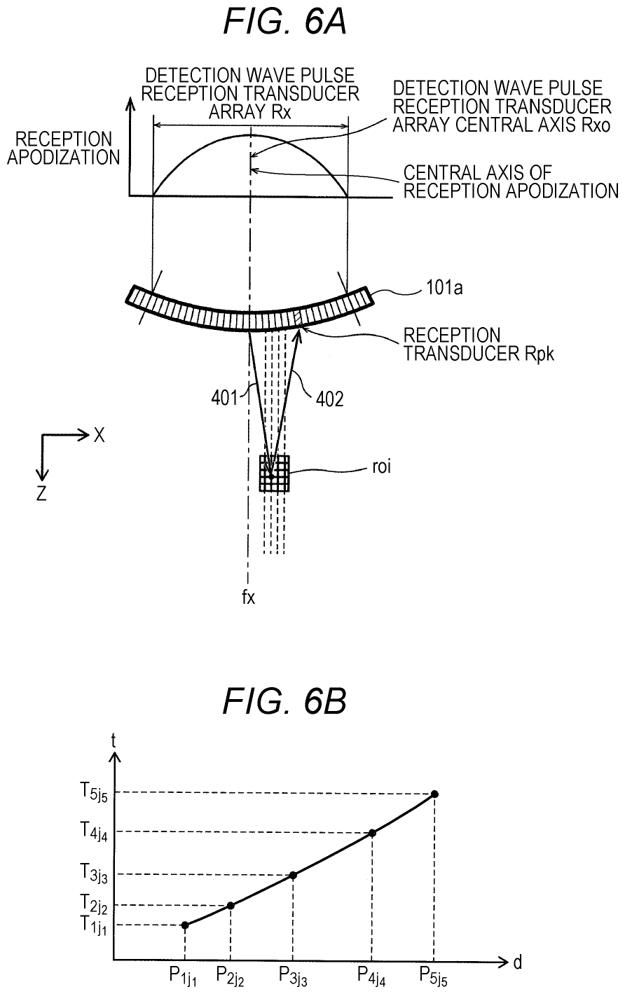

Next, as shown in the schematic view of FIG. 6B, the speed calculator 109 calculates the propagation speed vij of the shear wave from the peak times Tij and T(i+1) jᵢ₊₁ of the respective displacements of two observation points Pijᵢ and P(i+1) jᵢ₊₁ adjacent to each other in the traveling direction of the shear wave and calculates the representative value as the propagation speed of the shear wave in the region of interest roi. Examples of the representative value includes an average value and a median value. Note that the d-axis, the horizontal axis in FIG. 6B, is a distance axis indicating a traveling path of the shear wave.

Then, the speed calculator 109 generates elastic modulus data elf by associating the propagation speed with the region of interest roi and outputs the elastic modulus data elf to display controller 111.

7. B-Mode Image Generator 110

The B-mode image generator 110 is a circuit that generates a B-mode tomographic image from the sequence of the acoustic line signal frame data ds1.

The B-mode image generator 110 acquires one frame of the acoustic line signal frame data ds1 included in the sequence of the acoustic line signal frame data ds1. Then, the B-mode image generator 110 converts the acoustic line signal frame data ds1 into luminance signal frame data b11 by performing envelope detection and logarithmic compression and outputs the luminance signal frame data b11 to the display controller 111.

8. Display Controller 111

The display controller 111 is a circuit that generates a B-mode tomographic image or an image in which elastic modulus information is superimposed on the B-mode tomographic image and causes the display 113 to display the image.

The display controller 111 acquires the luminance signal frame data b11 from the B-mode image generator 110 and the elastic modulus data elf from the speed calculator 109, performs coordinate conversion, and generates a B-mode image or an image in which elastic modulus data is superimposed on the B-mode image.

<Operation of Ultrasonic Diagnostic Apparatus 100>

The operation of the integrated SWS sequence of the ultrasonic diagnostic apparatus 100 having the above configuration will be described.

1. Overview of Operation

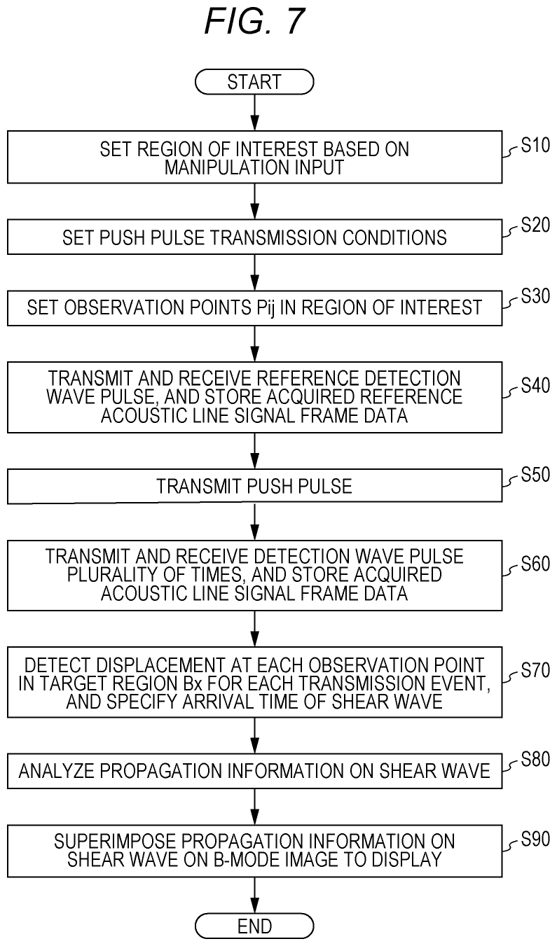

FIG. 7 is a flowchart showing the steps of the integrated SWS sequence in the ultrasonic diagnostic apparatus 100. The SWS sequence by the ultrasonic diagnostic apparatus 100 includes the steps of: setting a region of interest roi; performing reference detection wave transmission and reception to acquire the reference acoustic line signal frame data ds0 for extracting displacement caused by a shear wave for each subsequent transmission event; transmitting the push wave pulse ppp to transmit the push wave pp focused on a specific site FP in a subject to excite the shear wave in the subject; transmitting and receiving a detection wave pulse pwp1 to repeat, a plurality of times, transmission and reception of a detection wave pwp1 passing through a region of interest roi; and performing the propagation analysis of the shear wave to calculate a propagation speed of the shear wave and calculate an elastic modulus.

2. Operation of SWS Sequence

Hereinafter, the operation of the ultrasonic measurement processing of the elastic modulus after the B-mode image, in which the tissue is drawn based on the reflection components from the tissue of the subject based on a known method, is displayed on the display 113 will be described.

Note that the frame data of the B-mode image is generated as follows: the frame data of the acoustic line signals is generated in time series based on the reflection components from the tissue of the subject based on the transmission and reception of the ultrasonic waves performed by the transmission beam former 105 and the reception beam former 107 without the transmission of the push wave pulse ppp; the acoustic line signals are subjected to processing such as envelope detection and logarithmic compression to be converted into luminance signals; and the luminance signals are subjected to coordinate conversion into an orthogonal coordinate system. The details will be described later. The display controller 111 causes the display 113 to display the B-mode image in which the tissue of the subject is drawn.

First, in Step S10, a region of interest is set based on manipulation input from a user. More specifically, in a state where the B-mode image, which is a tomographic image of the subject acquired in real time by the probe 101, is displayed on the display 113, the controller 112 inputs the information designated by the manipulator from the manipulation inputter 102 and sets the region of interest roi representing an analysis target range in the subject with the position of the probe 101 as reference.

The designation of the region of interest roi by the manipulator is performed by, for example, displaying the latest B-mode image recorded in the data storage 108 on the display 113 and designating the region of interest roi through an inputter (not shown) such as a touch panel or a mouse. Herein, the region of interest roi is, for example, a fixed range away from the middle of the B-mode image in the array direction.

Next, in Step S20, the controller 112 sets the transmission conditions of the push pulse. Specifically, the push wave generator 103 acquires the information indicating the region of interest roi from the controller 112 and sets the position of the transmission focal point FP of the push wave pulse ppp and the push wave transmission transducer array Px. In this example, as shown in FIG. 3A, the push wave transmission transducer array Px is some of the plurality of transducers 101a. Moreover, the array direction transmission focal position fx coincides with an array direction center position we of the push wave transmission transducer array Px, and the depth direction transmission focal position fy is present in the vicinity of the region of interest roi. However, the positional relationship between the detection wave irradiation region Ax and the transmission focal point FP is not limited to the above and may be changed as appropriate depending on the form of the site of the subject to be examined or the like.

The information indicating the position of the transmission focal point FP and the push wave transmission transducer array Px is outputted to the transmission beam former 105 together with the pulse width PW and the application start time PT of the push wave pulse ppp as the transmission control signals.

Next, in Step S30, the observation points Pij are set in the region of interest. In this example, as shown in FIG. 6A, the observation points Pij are arranged at regular intervals in the z direction on the straight lines which extend in the z direction and pass the center of any of the reception transducers Rpk.

Next, in Step S40, the reference detection wave pulse is transmitted and received, and the acquired reference acoustic line signal frame data is stored. Specifically, a detection wave pulse is transmitted to the inside of the region of interest roi, and the acoustic line signal frame data are generated for the observation points Pij set in Step S30 and stored in the data storage 108 as the reference acoustic line frame data.

Next, in Step S50, a push pulse is transmitted. Specifically, the transmission beam former 105 generates the transmission profile based on the transmission control signals acquired from the push wave generator 103, including the information indicating the position of the transmission focal point FP and the push wave transmission transducer array Px, and the pulse width PW and the application start time PT of the push wave pulse ppp. The transmission profile includes the pulse signal sp and the delay time tpk for each transmission transducer included in the push wave transmission transducer array Px. Then, the push wave pulse ppp is supplied to each transmission transducer based on the transmission profile. Each transmission transducer transmits the pulsed push wave pp focused on a specific site in the subject.

Next, in Step S60, the detection wave pulse pwp1 is transmitted and received to and by the region of interest roi a plurality of times, and the acquired sequence of the acoustic line signal frame data ds1 is stored. Specifically, the transmission beam former 105 transmits the detection wave pulse pwp1 to the transducers included in the detection wave pulse transmission transducer array Tx toward the subject, and the reception beam former 107 generates the acoustic line signal frame data ds1 based on the reflected waves ec received by the transducers included in the detection wave pulse reception transducer array Rx. Immediately after the transmission of the push wave pp is finished, the above processing is repeated, for example, 10000 times per second. Accordingly, the acoustic line signal frame data ds1 of the inside of the region of interest roi is repeatedly generated from immediately after the occurrence of the shear wave until the propagation ends. The generated sequence of the acoustic line signal frame data ds1 is outputted to and stored in the data storage 108.

More specifically, the following processing is performed. First, the reception beam former 107 calculates, for an arbitrary observation point Pij present in the region of interest roi, the transmission time for the transmitted ultrasonic wave to arrive at the observation point Pij in the subject. Next, the reception beam former 107 sets the detection wave pulse reception transducer array Rx and calculates the reception times for the reflected detection wave from the observation points Pij to arrive at the respective reception transducers Rwk included in the detection wave pulse reception transducer array Rx. Then, the reception beam former 107 calculates a delay amount for each observation point Pij and for each reception transducer Rwk from the transmission time and the reception time and identifies the reception signal from the observation point Pij from the acoustic line signal frame data ds1 for each observation point Pij. Next, the reception beam former 107 weights and adds the reception signal identified for each observation point Pij to calculate an acoustic line signal for the observation point Pij. Herein, for the weighting, for example, reception apodization is performed so that the weighting of the transducer positioned at the center of the detection wave pulse reception transducer array Rx in the x direction becomes maximum. The reception beam former 107 stores the calculated acoustic line signal in the data storage 108.

Next, in Step S70, the displacement at each observation point Pij in the region of interest roi is detected for each transmission event, and the arrival time of the shear wave is specified. Specifically, in a first transmission event, for each observation point Pij, correlation processing is performed between the acoustic line signal frame data ds1 and the reference acoustic line signal frame data ds0 to detect the positional displacement amount for each observation point Pij. Furthermore, by performing this processing for all correlation events, the displacement amount for each transmission event is detected for each observation point Pij. Then, for each observation point Pij, a transmission event with the greatest displacement is specified, and the time at which the transmission event was performed is specified as a peak time.

Next, in Step S80, the propagation analysis of the shear wave is performed. Specifically, with the peak time for each observation point Pij specified in Step S70 as an index, two observation points Pij adjacent in the array direction are associated with each other, and the distance is divided by the time difference between the peak times to estimate the propagation speed of the shear wave. In the embodiment, as shown in FIG. 6B, for an observation point P1, an observation point P2, an observation point P3, an observation point P4 and an observation point P5 arranged in the array direction, the propagation path axis d of the shear wave is plotted on the horizontal axis, the peak times are plotted on the vertical axis. Then, the propagation speed of the shear wave is estimated by calculating the inclination between the observation points (=distance between the observation points/time difference between the peak times).

Finally, in Step S90, the propagation information on the shear wave is superimposed on the B-mode image to be displayed. Specifically, for example, the value of the elastic modulus is superimposed on the B-mode image. Note that the value of the elastic modulus may be displayed outside the B-mode image, or the propagation information on the shear wave may be superimposed on the B-mode image as color information. In another display mode, for example, information indicating the position, such as a symbol, an icon, or the like, is superimposed on the B-mode image, and the value of the elastic modulus at the indicated position is added to the outside of the B-mode image. Note that the display modes are not limited to these. For example, the elastic modulus may be displayed by dragging out a leader line from the position on the B-mode image to the outside of the B-mode image.

Thus, the processing of the SWS sequence shown in FIG. 7 ends. Through the above ultrasonic measurement processing of the elastic modulus, the elastic modulus data elf by the SWS sequence can be calculated.

3. Generation of B-Mode Image

The frame data of the B-mode image is generated as follows: the frame data of the acoustic line signals is generated in time series based on the reflection components from the tissue of the subject based on the transmission and reception of the ultrasonic waves performed by the transmission beam former 105 and the reception beam former 107 without the transmission of the push wave pulse ppp; the acoustic line signals are subjected to processing such as envelope detection and logarithmic compression to be converted into luminance signals; and the luminance signals are subjected to coordinate conversion into an orthogonal coordinate system. Herein, the operations themselves of the transmission, reception and phasing addition of ultrasonic waves are similar to the operations of the transmission, reception and phasing addition of the detection wave. Thus, the differences will be described below.

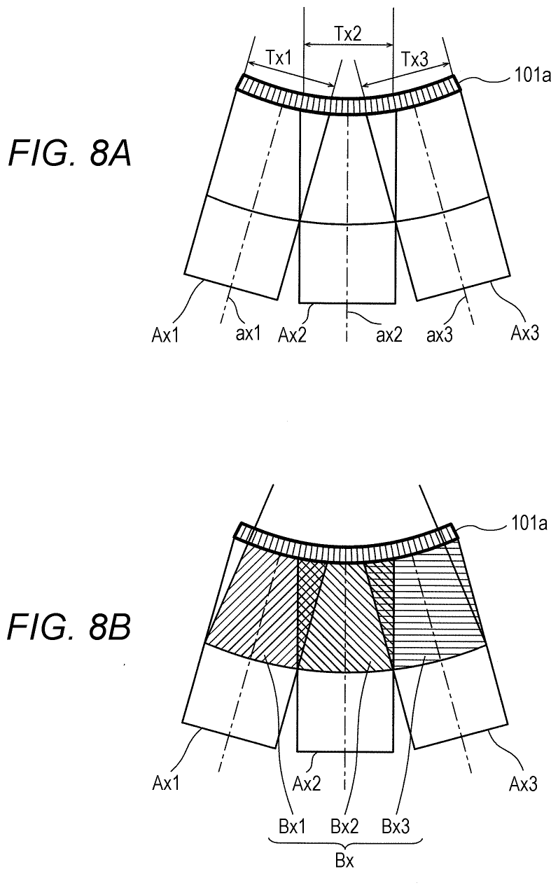

FIG. 8A is a schematic view showing a configuration overview for an ultrasonic pulse to create the frame data of the B-mode image. As shown in FIG. 8A, transmission transducer arrays Tx1, Tx2 and Tx3 are set for ultrasonic irradiation regions Ax1, Ax2 and Ax3, respectively such that plane waves having wavefronts orthogonal to the central axes ax1, ax2 and ax3 of the respective ultrasonic irradiation regions Ax1, Ax2 and Ax3 are sent out. Note that the ultrasonic irradiation regions Ax1, Ax2 and Ax3 are set such that any place that is not more than a predetermined distance from the surface of the transducer array 101a is included in at least one of the ultrasonic irradiation regions Ax1, Ax2 or Ax3. Note that the number of ultrasonic irradiation regions is not limited to three and may be any number.

FIG. 8B is a schematic view showing a target region Bx for creating the frame data of the B-mode image. As shown in FIG. 8B, the target region Bx, which is a target for creating an acoustic line signal, includes: a partial target region Bx1 included in the ultrasonic irradiation region Ax1; a partial target region Bx2 included in the ultrasonic irradiation region Ax2; and a partial target region Bx3 included in the ultrasonic irradiation region Ax3. Note that the target region Bx as a whole is defined as a set of places that are not more than a predetermined distance from the surface of the transducer array 101a. Note that the number of partial target regions is not limited to three and may be any number. In addition, the partial target regions Bx1, Bx2 and Bx3 partially overlap in FIG. 8B, but partial target regions may be set such that no regions overlap.

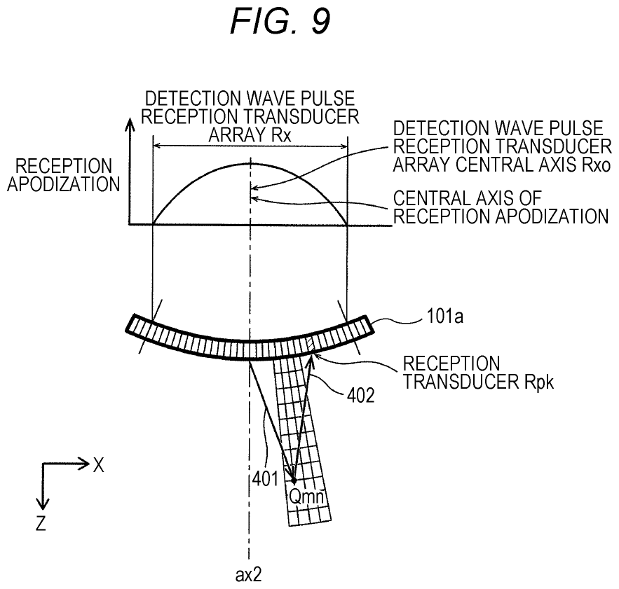

FIG. 9 shows an overview of reflected ultrasonic wave reception in the reception beamforming. In the processing of the B-mode image, as shown in FIG. 9, the acoustic line signal ds is generated by performing addition for all reception transducers Rpk after delay processing is performed on the reception signals rfk received by the reception transducers Rpk included in the detection wave pulse transmission transducer array Rx from a plurality of observation points Qmn included in the target region Bx. The observation points Qmn are arranged radially from the center of the circular arc where the transducer array 101a is arranged. Specifically, the observation points Qmn are arranged at regular intervals on straight lines that pass through one of a plurality of points provided at regular intervals on the surface of the transducer array 101a and are orthogonal to tangents to the surface of the transducer array 101a at the point. In other words, the observation points Qmn are arranged on the intersections of straight lines radiating from the center of the arc forming the surface of the transducer array 101a and arcs concentrically extending from the center of the circular arc. Note that each straight line preferably passes through the center of any of the transducers 101a and extends in the front direction of the transducers 101a. Since the width in the array direction of the range where the observation point Pij can exist does not exceed the width of the detection wave pulse reception transducer array Rx, but the range where the observation points Qmn can exist expands according to the depth, the range where the observation points Qmn can exist is wider than the range where the observation points Pij can exist. Meanwhile, the intervals between the observation points Qmn in the array direction (x direction) increase as the position in the depth direction (z direction) deepens, and the spatial resolution in the array direction decreases as the distance from the transducer array 101a increases.

The operation of creating the frame data of the B-mode image is as follows. First, the transmission beam former 105 transmits an ultrasonic wave to the ultrasonic irradiation region Ax1 as described above, and the reception beam former 107 generates the acoustic line signal for the observation point Qmn in the partial target region Bx1 described above. Next, the transmission beam former 105 transmits an ultrasonic wave to the ultrasonic irradiation region Ax1 described above, and the reception beam former 107 generates the acoustic line signal for the observation point Qmn in the partial target region Bx2 described above. Next, the transmission beam former 105 transmits an ultrasonic wave to the ultrasonic irradiation region Ax3 as described above, and the reception beam former 107 generates the acoustic line signal for the observation point Qmn in the partial target region Bx3 described above. Accordingly, the frame data of the acoustic line signal is generated. Then, the B-mode image generator 110 converts the acoustic line signal into a luminance signal frame data for each observation point Qmn by performing envelope detection and logarithmic compression on the acoustic line signal. Then, the display controller 111 converts the position of the observation point Qmn in the frame data of the luminance signal into an orthogonal coordinate system for display, and generates and displays a B-mode image. The method for creating the frame data of the B-mode image is not limited to the above, and the frame data of the image may be created by normal focus transmission.

<Summary>

With the above configuration, the distance between observation points in the array direction, which is the propagation direction of the shear wave, does not change regardless of the depth of the region of interest. Therefore, even if the region of interest is present at a deep position, it is possible to suppress a decrease in speed detection accuracy caused by an excessive distance between observation points in the array direction.

Moreover, in the above configuration, the detection wave is sent out as a plane wave in the z direction, which is the front direction of the transducer that is the center of the transmission transducer array Tx for the detection wave. Accordingly, in the ultrasonic irradiation region Ax, particularly in the vicinity of the center in the array direction, the front direction of the transducer and the vibration direction of the plane wave coincide with each other. Thus, the amplitude of the ultrasonic wave is large, and a highly accurate acoustic line signal can be generated.

### Modification 1

As described above, the width of the B-mode image in the array direction increases in accordance with the depth, whereas the width in the array direction of the range where the observation point Pij can exist is constant regardless of the depth. Therefore, the range in which the B-mode image can be generated is wider than the range in which the region of interest roi can be set. In the embodiment, the detection wave is transmitted and received such that the transducer at the array direction center position of the transducer array 101a is the center of the array. However, with this configuration, there may be a region where the region of interest roi cannot be set in a place where the depth is long and far from the center of the image although the B-mode image is acquired.

In Modification 1, the region of interest roi can be set at any place within the region where the B-mode image can be acquired.

<Transmission and Reception Control of Detection Wave>

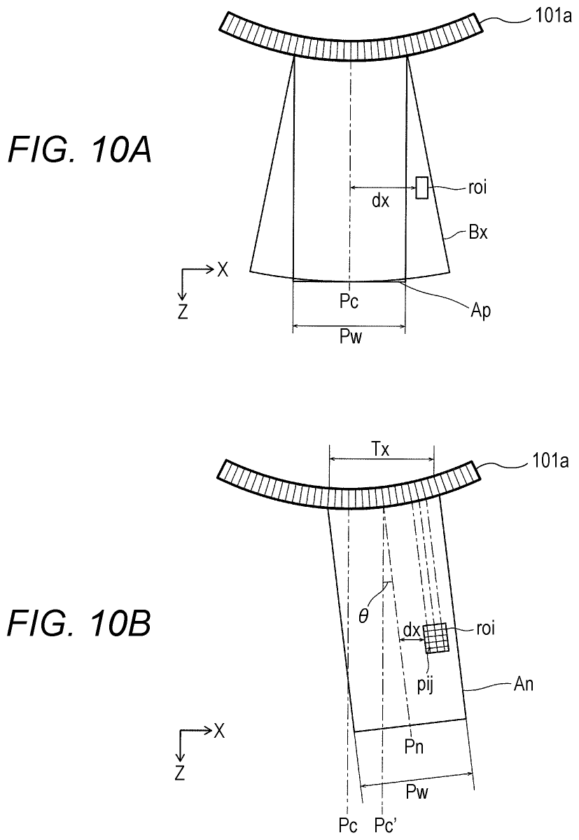

FIG. 10A is a schematic view showing the relationship between a region of interest roi and an ultrasonic irradiation region Ap of a detection wave when the transducer at the array direction center position of a transducer array 101a is the center of the array.

As shown in FIG. 10A, the ultrasonic irradiation region Ap of the detection wave has a width Pw with a central axis Pc, which passes through the array direction center position of the transducer array 101a and extends in the z direction, as the central axis. Since an observation point needs to be set within the ultrasonic irradiation region Ap of the detection wave, the measurable range in which the observation point can be set is the entire region inside the ultrasonic irradiation region Ap of the detection wave. On the other hand, since a target region Bx of a B-mode image is wider than the ultrasonic irradiation region Ap, which is a measurable range in the array direction, the region of interest roi set based on the B-mode image does not exist inside the measurable range in some cases. Specifically, when an array direction distance dx between the region of interest roi and the central axis Pc meets dx>Pw/2 with respect to the width Pw of the ultrasonic irradiation region Ap, the region of interest roi does not exist in the measurable range, and the observation point Pij cannot be set.

FIG. 10B is a schematic view showing a transmission and reception region of a detection wave in Modification 1. As shown in FIG. 10B, a detection wave generator sets a detection wave pulse transmission transducer array Tx so as to pass through the entire region of interest roi. Specifically, the ultrasonic irradiation region An having the width Pw is set with, as the central axis, a central axis Pn which passes through the center of the circular arc forming the surface of the transducer array 101a and forms an angle θ with respect to the z direction (the central axis Pc and Pc′ parallel to the central axis Pc). Accordingly, the inside of the ultrasonic irradiation region An becomes the measurable range. Note that the central axis Pn passes through the surface of a transducer Rh positioned at the middle of the detection wave pulse transmission transducer array Tx, and the central axis ph is orthogonal to the tangent to the transducer array 101a at the transducer Rh. At this time, transmission beamforming is performed such that a detection wave pw becomes a plane wave propagating in the direction that the central axis Pn extends. That is, in Modification 1, the same transmission beam forming as in Embodiment 1 is performed while the transducer Rh is regarded as the transducer at the array direction center position of the transducer array 101a.

And, in reception beamforming, as shown in FIG. 10B, observation points Pij are arranged at regular intervals on a plurality of straight lines parallel to the central axis Pn. Specifically, the observation points Pij are arranged at the intersections of the plurality of straight lines parallel to the central axis Pn and straight lines orthogonal to the central axis Pn. Note that each of the straight lines parallel to the central axis Pn may be a curve passing through the center of any of reception transducers Rpk.

Note that a transmission focal point FP of a push pulse may also be moved onto the central axis Pn. Specifically, a position, which is on the central axis Pn and has the same depth as the region of interest roi, is set as the transmission focal point FP of the push pulse. Also in the transmission beamforming of the push pulse, the transducer Rh on the central axis Pn may be the middle of the transmission transducer array, and the push pulse may be transmitted along the central axis Pn. Note that, in the case where the push pulse is transmitted along the central axis Pn, the vibration direction of the shear wave is parallel to the central axis Pn so that the observation points are preferably provided on straight lines orthogonal to the central axis Pn.

<Summary>

Even with the above configuration, the distance between observation points in the array direction, which is the propagation direction of the shear wave, does not change regardless of the depth of the region of interest. Therefore, even if the region of interest is present at a deep position, it is possible to suppress a decrease in speed detection accuracy caused by an excessive distance between observation points in the array direction.

Moreover, with the above configuration, even if the region of interest does not exist in the vicinity of the transducer at the center of the transmission transducer array Tx for the detection wave, in the z direction, the entire region of interest can be made present in the ultrasonic irradiation region. Therefore, the propagation analysis of the shear wave can be conducted in a region where a B-mode image can be acquired, even at an absent position in the z direction from the transducer that is the center of the transmission transducer array Tx for the detection wave.

Furthermore, in the above configuration, the transmission transducer array Tx is set such that the region of interest exists in the vicinity of front direction of the transducer at the center of the transmission transducer array Tx for the detection wave. Accordingly, in the ultrasonic irradiation region An, particularly, in the vicinity of the front direction of the transducer at the center of the transmission transducer array Tx, the amplitude of the ultrasonic wave is large, and a highly accurate acoustic line signal can be generated.

Furthermore, in the above configuration, the transmission focal point FP of the push pulse is set in the front direction of the transducer at the center of the transmission transducer array Tx for the detection wave. Therefore, since the region of interest and the transmission focal point FP of the push pulse can be brought close to each other without becoming excessively close, the accuracy of propagation analysis can be improved by setting the amplitude of the shear wave in the region of interest to be large.

### Modification 2

As described above in Modification 1, the range in which the B-mode image can be generated is wider than the range in which the region of interest roi can be set. In Modification 1, the position of the transmission transducer array Tx is moved for the transmission and reception of the detection wave, but the following control is also possible.

Also in Modification 2, a region of interest roi can be set at any place within the region where the B-mode image can be acquired.

<Transmission and Reception Control of Detection Wave>

In this modification, it is determined whether or not the region of interest roi exists inside an ultrasonic irradiation region Ap of a detection wave when the transducer at the array direction center position of a transducer array 101a is the center of the array. Specifically, as shown in FIG. 10A, it is determined whether or not the region of interest roi is included in the ultrasonic irradiation region Ap of the detection wave when the transducer at the array direction center position of the transducer array 101a is the center of the array. Then, when the entire region of interest roi is included in the ultrasonic irradiation region Ap, as described in the embodiment, the detection wave is transmitted so that the plane wave propagates in the ultrasonic irradiation region Ap in the z direction, and observation points Pij are arranged at regular intervals in the z direction on straight lines which extend in the depth direction (z direction) and are parallel to each other. On the other hand, when the whole or part of the region of interest roi is not included in the ultrasonic irradiation region Ap, the detection wave is transmitted and received by a similar method for the acquisition of the acoustic line for creating the B-mode image. Specifically, as shown in FIG. 8A, ultrasonic irradiation regions Ax1, Ax2 and Ax3 are set so that a plane wave having a wavefront orthogonal to central axes ax1, ax2 and ax3 of respective ultrasonic irradiation regions Ax1, Ax2 and Ax3 is sent out, and a detection wave is transmitted to the ultrasonic irradiation region including the region of interest roi. Then, as shown in FIG. 9, observation points Pij are arranged radially from the center of the circular arc where the transducer array 101a is arranged. Note that the observation points Pij may be set so that the observation points Pij adjacent in the x direction are at the same positions (depth) in the z direction.

Note that, if the region of interest roi extends over two of the ultrasonic irradiation regions Ax1, Ax2 and Ax3, the following operation may be repeated: a detection wave is transmitted to any one of the ultrasonic irradiation regions Ax1, Ax2 and Ax3; thereafter, the reception from the observation point included in the region of interest in the ultrasonic irradiation region is performed; a detection wave is transmitted to one of the other two regions; and thereafter the reception from the observation point included in the region of interest in the ultrasonic irradiation region is performed. More specifically, when the region of interest roi extends over the two ultrasonic irradiation regions Ax1 and Ax2, the transmission and reception are performed as follows. First, after a detection wave has been transmitted to the ultrasonic irradiation region Ax1, the reflected detection wave is received, and an acoustic line signal is generated for the observation point Pij set within the region where the region of interest roi and the ultrasonic irradiation region Ax1 overlap. Subsequently, after a detection wave has been transmitted to the ultrasonic irradiation region Ax2, the reflected detection wave is received, and an acoustic line signal is generated for the observation point Pij set within the region where the region of interest roi and the ultrasonic irradiation region Ax2 overlap. The transmission and reception of the detection waves are performed alternately to perform the transmission and reception of the detection wave for the entire region of interest roi.

<Summary>

Even with the above configuration, the distance between observation points in the array direction, which is the propagation direction of the shear wave, does not change regardless the depth of the region of interest when the region of interest exists in the vicinity of the front direction of the transducer positioned at the middle of the transducer array of the ultrasonic probe. Therefore, even if the region of interest is present at a deep position, it is possible to suppress a decrease in speed detection accuracy caused by an excessive distance between observation points in the array direction.

Moreover, even with the above configuration, the propagation analysis of the shear wave can be performed in a region where a B-mode image can be acquired, even at an absent position in the z direction from the transducer at the center of the transmission transducer array Tx for the detection wave.

Furthermore, in the above configuration, when the region of interest is positioned distant from the transmission focal point FP of the push pulse, the observation points are radially arranged suitably for a convex probe as in the acquisition of the acoustic line signal related to the B-mode image. Since the amplitude of the shear wave is small at a position distant from the region of interest, and the improvement of the accuracy of propagation analysis is limited, the speed detection accuracy hardly decreases even in the above-described processing. Therefore, it is possible to improve the speed detection accuracy of the shear wave at a place where the improvement is expected, and the accuracy improvement can be made efficient.

### Modification 3

In Modifications 1 and 2, when the region of interest roi is not inside the ultrasonic irradiation region Ap of the detection wave in a case where the transducer at the array direction center position of the transducer array 101a is the center of the array, the region of interest roi can be set by moving the position of the transmission transducer array Tx or setting the observation points as in the acquisition of the acoustic line signal related to the B-mode image.

Modification 3 includes a configuration that provides an interface which allows a user to select any one of Modification 1 or Modification 2 for use.

<Operation>

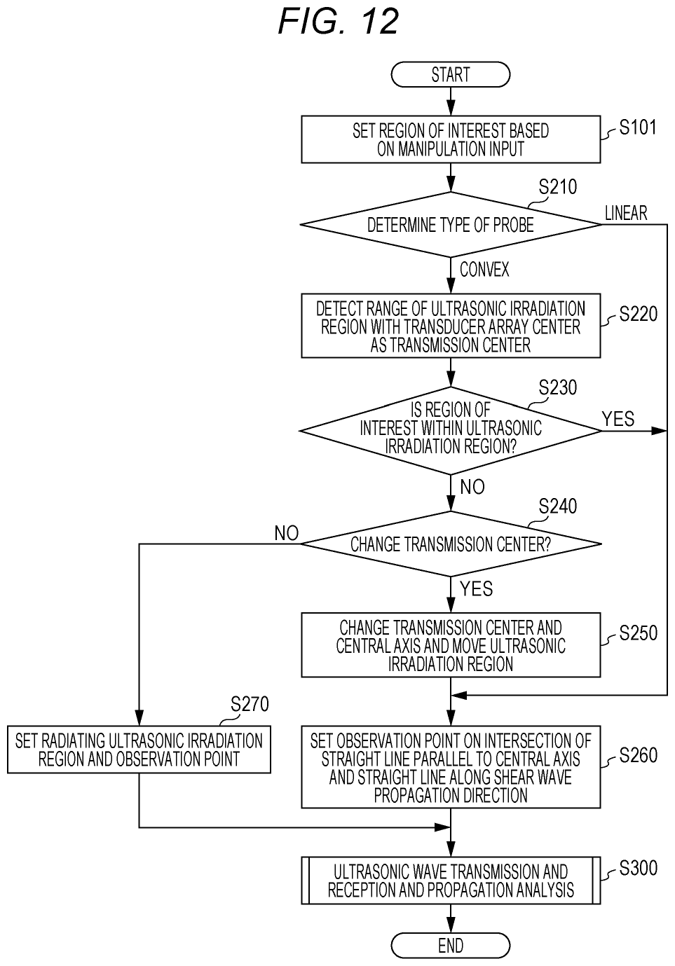

FIG. 12 is a flowchart showing steps of an integrated SWS sequence according to Modification 3.

First, in Step S101, a region of interest is set based on manipulation input from a user. More specifically, in a state where a B-mode image, which is a tomographic image of a subject acquired in real time by a probe 101, is displayed on a display 113, a controller 112 inputs information designated by the manipulator from a manipulation inputter 102 and sets the region of interest roi representing an analysis target range in the subject with the position of the probe 101 as reference. At this time, when the region of interest roi does not exist inside an ultrasonic irradiation region Ap of a detection wave in a case where the transducer at the array directional center position of the transducer array 101a is the center of the array, input as to whether or not to move the position of a transmission transducer array Tx is also accepted for the transmission and reception of the detection wave as in Modification 1.

Next, in Step S210, a controller of an ultrasonic diagnostic apparatus determines the type of the connected ultrasonic probe. When the ultrasonic probe is a linear probe, the processing proceeds to Step S260. On the other hand, when the ultrasonic probe is a convex probe, the processing proceeds to Step S220.

When the ultrasonic probe is a convex probe, in Step S220, the controller of the ultrasonic diagnostic apparatus detects the ultrasonic irradiation region of the detection wave when the transducer at the array direction center position of the transducer array is the center of the array. As shown in FIGS. 10A and 10B, the ultrasonic irradiation region of the detection wave has a width Pw with a central axis Pc, which passes through the array direction center position of the transducer array and extends in the z direction, as the central axis.

Next, in Step S230, the controller of the ultrasonic diagnostic apparatus determines whether or not the entire region of interest roi is included in the ultrasonic irradiation region detected in Step S220. When the entire region of interest roi is included in the ultrasonic irradiation region, the processing proceeds to Step S260. On the other hand, when part or whole of the region of interest roi is not included in the ultrasonic irradiation region, the processing proceeds to Step S240.

Next, in Step S240, the controller of the ultrasonic diagnostic apparatus determines whether or not to move the position of the transmission transducer array Tx for the transmission and reception of the detection wave. When the input as to move the position of the transmission transducer array Tx has been obtained in Step S101, the processing proceeds to Step S250. On the other hand, when the input as to not move the position of the transmission transducer array Tx has been obtained in Step S101, the processing proceeds to Step S270.

Next, in Step S250, the controller of the ultrasonic diagnostic apparatus changes the center of the transmission transducer array Tx and the central axis of the ultrasonic irradiation region for the transmission and reception of the detection wave. Specifically, as described above in Modification 1, the ultrasonic irradiation region An having the width Pw is set with, as the central axis, a central axis Pn which passes through the center of the circular arc forming the surface of the transducer array so as to pass through the entire region of interest roi and forms an angle θ with respect to the z direction.

Next, in Step S260, a phasing adder of the ultrasonic diagnostic apparatus sets an observation point in the region of interest roi. Specifically, the phasing adder of the ultrasonic diagnostic apparatus sets the observation point on an intersection of a straight line parallel to the central axis of the ultrasonic irradiation region and a straight line extending in the x direction. Therefore, when the central axis of the ultrasonic irradiation region has been changed in Step S250, as shown in FIG. 10B, an observation point is set on an intersection of a straight line forming an angle θ with respect to the z direction and a straight line forming an angle θ with respect to the x direction. Meanwhile, when it has been determined in Step S210 that the probe is a linear probe or when it has been determined in Step S230 that the region of interest roi exists in the ultrasonic irradiation region of the detection wave in a case where the transducer at the array direction center position of the transducer array is the center of the array, the central axis of the ultrasonic irradiation region is a straight line extending in the z direction so that the observation point is set on an intersection of the straight line extending in the z direction and the straight line extending in the x direction.

In Step S270, the phasing adder of the ultrasonic diagnostic apparatus sets an observation point in the region of interest roi. Specifically, the phasing adder of the ultrasonic diagnostic apparatus sets observation points on intersections of straight lines extending radially from the center of the arc forming the surface of the ultrasonic probe and circular arcs concentrically extending from the center as in the acquisition of the acoustic line signal for generating a B-mode image.

In Step S300, transmission of a push pulse, subsequent transmission and reception of a detection wave, and propagation analysis of a shear wave are performed. The details are the same as Steps S20 to S90 according to the embodiment except that the transmission and reception profile of the detection wave has already been decided, and thus detailed description will be omitted.

<Summary>

Even with the above configuration, the distance between observation points in the array direction, which is the propagation direction of the shear wave, does not change regardless the depth of the region of interest when the region of interest exists in the vicinity of the front direction of the transducer positioned at the middle of the transducer array of the ultrasonic probe. Therefore, even if the region of interest is present at a deep position, it is possible to suppress a decrease in speed detection accuracy caused by an excessive distance between observation points in the array direction.

Moreover, according to the above configuration, when the region of interest does not exist in the vicinity of the front direction of the transducer positioned at the middle of the transducer array of the ultrasonic probe, the selection is possible as to change the transmission direction of the detection wave or as to perform the transmission and reception of the detection wave as in the transmission and reception of ultrasonic waves for generating a B-mode image. Therefore, to improve the accuracy of the propagation analysis of the shear wave, the transmission direction of the detection wave is changed, while the detection wave can be transmitted and received under the same conditions as the transmission and reception of the ultrasonic waves for generating a B-mode image for association with the B-mode image. Thus, utilization according to the application is possible.

### Other Modifications According to Embodiments

(1) In Embodiments and each Modification, the distance between the observation points in the propagation direction of the shear wave is constant regardless of the distance between the region of interest roi and the probe. However, for example, the distance between the observation points in the propagation direction of the shear wave may be decreased as the distance between the region of interest roi and the probe is increased. Specifically, for example, the observation point may be provided on a straight line extending radially from the point deeper than the deepest part in the B-mode image to each transducer. Even with this configuration, them is no location where the distance between the observation points in the propagation direction of the shear wave becomes excessively long. Thus, it is possible to obtain the effect of suppressing the decrease in the spatial resolution and the effect of suppressing the insufficiency of the number of observation points.

(2) In Embodiments and each Modification, the plurality of observation points are provided in the depth direction in the region of interest roi. However, for example, a plurality of observation points may be aligned only in the array direction in the region of interest roi, not in the depth direction. In this case, the plurality of observation points may be set, for example, at the same depth. Alternatively, for example, the plurality of observation points may be aligned in a direction orthogonal to the transmission central axis of the push pulse, that is, in a direction orthogonal to the pressing direction by the push pulse. Accordingly, the propagation analysis of the shear wave can be further simplified, and the amount of arithmetic operation can be reduced.

(3) In each Modification, the method of transmitting the push pulse and the method of transmitting the detection wave are changed when the method of setting the observation points in the region of interest roi is changed. However, for example, the method of transmitting the push pulse may be the same as Embodiments, and the method of transmitting the detection wave may be the same as Embodiments. Moreover, one or both of the method of transmitting the push pulse and the method of transmitting the detection wave may be a known method different from Embodiments and Modifications, and the same effects can be obtained if the method of setting the observation points in the reception of the detection wave is as described above.

(4) In Embodiments, the ultrasonic diagnostic apparatus 100 performs the step of the reference detection wave pulse transmission and reception before the step of the push wave pulse transmission, and the displacement detector detects the displacement Ptij at the observation point Pij based on the difference between the acoustic line signal frame data ds1 and the reference acoustic line signal frame data ds0 formed by the transmission and reception of the reference detection wave pulse. However, the method of detecting the amount of tissue displacement is not limited to this case. For example, the ultrasonic diagnostic apparatus does not perform the step of the reference detection wave pulse transmission and reception and does not generate the reference acoustic line signal frame data ds0. Then, based on the difference between the acoustic line signal frame data ds1 and the acoustic line frame data ds(1-1) obtained in the transmission event one before, the displacement detector detects a change amount ΔPtij of the displacement Ptij at the observation point Pij between the transmission events. Then, for each observation point Pij, the displacement Ptij at the observation point Pij may be generated by integrating the change amount ΔPtij of the displacement Ptij between the plurality of transmission events. Note that the detection of the change amount ΔPtij between the transmission events is not limited to between two consecutive transmission events. From the difference between any two acoustic line signal frame data ds1, the change amount ΔPtij of the displacement Ptij at the observation point Pij may be calculated.

(5) For the ultrasonic diagnostic apparatus according to Embodiments and each Modification, all or some constituents thereof may be realized by a single-chip or multiple-chip integrated circuit, by a computer program, or by any other form. For example, the push wave generator and the detection wave generator may be realized by one chip. The reception beam former may be realized by one chip, and the speed detector and the like may be realized by another chip.

When the constituents are realized by an integrated circuit, the constituents are typically realized as a large scale integration (LSI). Herein, the LSI is used, but the constituents may be called an IC, a system LSI, a super LSI or an ultra LSI depending on the degree of integration.

In addition, the method of circuit integration is not limited to LSI, and may be realized by a dedicated circuit or a general-purpose processor. After the LSI is manufactured, a field programmable gate array (FPGA) that is programmable, or a reconfigurable processor capable of reconfiguring connection and setting of circuit cells inside the LSI may be used.

Furthermore, if an integrated circuit technology that replaces the LSI emerges due to the advancement of the semiconductor technology or another derived technology, the functional blocks may be integrated by using the technology as matter of course.

Further, the ultrasonic diagnostic apparatus according to each Embodiment and each Modification may be realized by a program written in a storage medium and a computer that reads and executes the program. The storage medium may be any recording medium such as a memory card and a CD-ROM. In addition, the ultrasonic diagnostic apparatus according to the present invention may be realized by a program downloaded via a network and a computer which downloads the program from the network to execute.

(6) All the embodiments described above show preferred specific examples of the present invention. Numerical values, shapes, materials, constituents, arrangement positions and connection forms of constituents, steps, order of steps, and the like shown in the embodiments are merely examples, and are not intended to limit the present invention. Moreover, among the constituents in the embodiments, steps not described in the independent claims that indicate the highest concept of the present invention are described as arbitrary constituents that constitute a more preferable embodiment.

Furthermore, for easy understanding of the present invention, the scales of the constituents in each of the drawings mentioned in each of the above embodiments may be different from actual ones. Further, the present invention is not limited by the description of each of the above embodiments and can be changed as appropriate without departing from the gist of the present invention.

Furthermore, in the ultrasonic diagnostic apparatus, there are also members such as circuit components and lead wires on a board, but various aspects can be implemented based on ordinary knowledge in the art of electric wiring and electric circuits and are not directly relevant as the description of the present invention so that the description is omitted. Note that each of the drawings shown above is a schematic diagram, and is not necessarily strictly illustrated.

<<Supplement>>

(1) An ultrasonic signal processing apparatus according to an embodiment is an ultrasonic signal processing apparatus that excites a shear wave in a subject to analyze a propagation state of the shear wave by using a convex ultrasonic probe, the ultrasonic signal processing apparatus including: a push wave transmitter that causes the ultrasonic probe to transmit a push wave for causing displacement in the subject; a detection wave transmitter that causes the ultrasonic probe to transmit a detection wave after the transmission of the push wave, the detection wave passing through a region of interest which indicates an analysis target range in the subject; a detection wave receiver that receives an ultrasonic wave reflected from the region of the interest by using the ultrasonic probe and converts the ultrasonic wave into a reception signal, the ultrasound corresponding to the detection wave; a phasing adder that sets a plurality of observation points in the region of the interest and performs phasing addition for each of the plurality of the observation points to generate an acoustic line signal; and a mechanical property calculator that calculates a mechanical property of the subject in the region of the interest based on the acoustic line signal for each of the plurality of the observation points, in which a distance between observation points along a propagation direction of the shear wave in the region of the interest is set to be not more than a distance between observation points along the propagation direction of the shear wave when a region closer to the ultrasonic probe than the region of the interest is set as a region of the interest.

Moreover, an ultrasonic signal processing method according to an embodiment is an ultrasonic signal processing method that excites a shear wave in a subject to analyze a propagation state of the shear wave by using a convex ultrasonic probe, the ultrasonic signal processing method including: causing the ultrasonic probe to transmit a push wave for causing displacement in the subject; causing the ultrasonic probe to transmit a detection wave after the transmission of the push wave, the detection wave passing through a region of interest which indicates an analysis target range in the subject; receiving an ultrasonic wave reflected from the region of the interest by using the ultrasonic probe and converting the ultrasonic wave into a reception signal, the ultrasonic wave corresponding to the detection wave; setting a plurality of observation points so that a distance between observation points along a propagation direction of the shear wave in the region of the interest is set to be not more than a distance between observation points along the propagation direction of the shear when a region closer to the ultrasonic probe than the region of the interest is set as a region of the interest; performing phasing addition for each of the plurality of the observation points to generate an acoustic line signal; and calculating a mechanical property of the subject in the region of the interest based on the acoustic line signal for each of the plurality of the observation points.

Furthermore, a program according to an embodiment is a program causing a computer to execute ultrasonic signal processing that excites a shear wave in a subject to analyze a propagation state of the shear wave by using a convex ultrasonic probe, the ultrasonic signal processing including: causing the ultrasonic probe to transmit a push wave for causing displacement in the subject; causing the ultrasonic probe to transmit a detection wave following the transmission of the push wave, the detection wave passing through a region of interest which indicates an analysis target range in the subject; receiving ultrasound reflected from the region of the interest by using the ultrasonic probe and converting the ultrasound into a reception signal, the ultrasound corresponding to the detection wave; setting a plurality of observation points so that a distance between observation points along a propagation direction of the shear wave in the region of the interest is set to be not more than a distance between observation points along a propagation direction of a shear when a region closer to the ultrasonic probe than the region of the interest is set as the region of the interest; performing phasing addition for each of the plurality of the observation points to generate an acoustic line signal; and calculating a mechanical property of the subject in the region of the interest based on the acoustic line signal for each of the plurality of the observation points.

According to the present disclosure, the spatial resolution does not decrease at a deep portion since the distance between the observation points in the propagation direction of the shear wave does not increase even when the distance between the observation points and the probe increases. Therefore, it is possible to suppress a decrease in the accuracy of the propagation speed of the shear wave due to the positional relationship between a region of interest and the probe.

(2) Moreover, in the ultrasonic signal processing apparatus according to (1), the phasing adder may set the plurality of the observation points on a plurality of straight lines which are parallel to each to other and exist in the region of the interest.

According to the above configuration, the distance between the observation points in the propagation direction of the shear wave does not depend on the distance between the observation points and the probe. Thus, the above-described effects can be securely obtained with simple configuration.

(3) Furthermore, in the ultrasonic signal processing apparatus according to (2), the plurality of the straight lines may be orthogonal to tangents to a surface of the ultrasonic probe at a center position of a transmission transducer array used for the transmission of the detection wave.

According to the above configuration, the observation points are provided in a direction intersecting the propagation direction of the detection wave. Thus, the propagation analysis of the shear wave can be efficiently conducted.

(4) Further, in the ultrasonic signal processing apparatus according to (2) or (3), each of the plurality of the straight lines may pass in the vicinity of the center of each transducer existing on the surface of the ultrasonic probe.

According to the above configuration, the arithmetic operation of the acoustic line signal can be calculated with each transducer as reference. Thus, it is possible to efficiently perform the phasing addition as well as improve the SNR.

(5) Moreover, in the ultrasonic signal processing apparatus according to any one of (1) to (4), the detection wave transmitter may transmit the detection wave with a transducer on the ultrasonic probe closest to a point close to the region of the interest as the center position of the transmission transducer array used for the transmission of the detection wave.

According to the above configuration, it is possible to emit a detection wave with sufficient intensity into the region of interest and improve the intensity and SNR of the acoustic line signal.

(6) Furthermore, the ultrasonic signal processing apparatus according to any one of (1) to (5) may further include a measurement range determiner that decides a measurable range, which indicates a range in which the observation points can be set, according to a position of the transmission transducer array used for the transmission of the detection wave by the detection wave transmitter.

According to the above configuration, when the observation points are set on the basis of the transmission transducer array, it is possible to prevent the observation points from being not set in the region of interest.

(7) Further, in the ultrasonic signal processing apparatus according to (6), the phasing adder may change one or more positions of the plurality of the observation points in a case where the region of the interest is not included in the measurable range so that a distance between the plurality of the observation points in a direction along one of tangents to a surface of the ultrasonic probe increases according to a distance between the observation points and the ultrasonic probe.

According to the above configuration, to set the observation points on the basis of the transmission transducer array, observation points can be set by a different method when a situation occurs in which an observation point is not set in the region of interest.

(8) Moreover, the ultrasonic signal processing apparatus according to (6) or (7), the phasing adder may change one or more positions of the plurality of the observation points in a case where the region of the interest extends over an inside and an outside of the measurable range so that a distance between the plurality of the observation points in a direction along one of tangents to a surface of the ultrasonic probe increases according to a distance between the observation points and the ultrasonic probe.

According to the above configuration, to set the observation points on the basis of the transmission transducer array, observation points can be set by a different method when a region occurs in which an observation point is not set in the region of interest.

(9) Furthermore, the ultrasonic signal processing apparatus according to (6) may further include an inputter that accepts selection from a user as to perform processing of transmitting the detection wave with a transducer on the ultrasonic probe closest to a point close to the region of the interest as a center position of the transmission transducer array used for the transmission of the detection wave or of changing, by the phasing adder, one or more positions of the plurality of the observation points so that a distance between the plurality of the observation points in a direction along one of tangents to a surface of the ultrasonic probe increases according to a distance between the observation points and the ultrasonic probe, in a case where at least part of the region of the interest is not included in the measurable range.

According to the above configuration, to set observation points on the basis of the transmission transducer array, the user can select how observation points are set when a region occurs in which an observation point is not set in the region of interest.

An ultrasonic diagnostic apparatus and an ultrasonic signal processing method according to the present disclosure are useful for measuring mechanical properties of a subject by using ultrasonic waves. Thus, it is possible to improve the measurement accuracy of the mechanical properties of a tissue or a substance, and the ultrasonic diagnostic apparatus and the ultrasonic signal processing method according to the present disclosure have high applicability in medical diagnostic device, nondestructive examination apparatus, and the like.

Although embodiments of the present invention have been described and illustrated in detail, the disclosed embodiments are made for purposes of illustration and example only and not limitation. The scope of the present invention should be interpreted by terms of the appended claims

What is claimed is:

1. An ultrasonic signal processing apparatus that excites a shear wave in a subject to analyze a propagation state of the shear wave by using a convex ultrasonic probe, the ultrasonic signal processing apparatus comprising:
a push wave transmitter that causes the ultrasonic probe to transmit a push wave for causing displacement in a subject;
a detection wave transmitter that causes the ultrasonic probe to transmit a detection wave after the transmission of the push wave, the detection wave passing through a region of interest which indicates an analysis target range in the subject;
a detection wave receiver that receives an ultrasonic wave reflected from the region of the interest by using the ultrasonic probe and converts the ultrasonic wave into a reception signal, the ultrasound corresponding to the detection wave;
a phasing adder that sets a plurality of observation points in the region of the interest and performs phasing addition for each of the plurality of the observation points to generate an acoustic line signal; and
a mechanical property calculator that calculates a mechanical property of the subject in the region of the interest based on an acoustic line signal for each of the plurality of the observation points,
wherein a distance between observation points along a propagation direction of a shear wave in the region of the interest is set to be not more than a distance between observation points along a propagation direction of a shear wave when a region closer to the ultrasonic probe than the region of the interest is set as the region of the interest.

2. The ultrasonic signal processing apparatus according to claim 1, wherein the phasing adder sets the plurality of the observation points on a plurality of straight lines which are parallel to each to other and exist in the region of the interest.

3. The ultrasonic signal processing apparatus according to claim 2, wherein the plurality of the straight lines are orthogonal to tangents to a surface of the ultrasonic probe at a center position of a transmission transducer array used for the transmission of the detection wave.

4. The ultrasonic signal processing apparatus according to claim 2, wherein each of the plurality of the straight lines passes in a vicinity of a center of each transducer existing on a surface of the ultrasonic probe.

5. The ultrasonic signal processing apparatus according to claim 1, wherein the detection wave transmitter transmits the detection wave with a transducer on the ultrasonic probe closest to a point close to the region of the interest as a center position of a transmission transducer array used for the transmission of the detection wave.

6. The ultrasonic signal processing apparatus according to claim 1, further comprising a measurement range determiner that decides a measurable range, which indicates a range in which the observation points can be set, according to a position of a transmission transducer array used for the transmission of the detection wave by the detection wave transmitter.

7. The ultrasonic signal processing apparatus according to claim 6, wherein the phasing adder changes one or more positions of the plurality of the observation points in a case where the region of the interest is not included in the measurable range so that a distance between the plurality of the observation points in a direction along one of tangents to a surface of the ultrasonic probe increases according to a distance between the observation points and the ultrasonic probe.

8. The ultrasonic signal processing apparatus according to claim 6, wherein the phasing adder changes one or more positions of the plurality of the observation points in a case where the region of the interest extends over an inside and an outside of the measurable range so that a distance between the plurality of the observation points in a direction along one of tangents to a surface of the ultrasonic probe increases according to a distance between the observation points and the ultrasonic probe.

9. The ultrasonic signal processing apparatus according to claim 6, further comprising an inputter that accepts selection from a user as to perform processing of transmitting the detection wave with a transducer on the ultrasonic probe closest to a point close to the region of the interest as a center position of the transmission transducer array used for the transmission of the detection wave or of changing, by the phasing adder, one or more positions of the plurality of the observation points so that a distance between the plurality of the observation points in a direction along one of tangents to a surface of the ultrasonic probe increases according to a distance between the observation points and the ultrasonic probe, in a case where at least part of the region of the interest is not included in the measurable range.

10. An ultrasonic diagnostic apparatus comprising:
a convex ultrasonic probe; and
the ultrasonic signal processing apparatus according to claim 1.

11. An ultrasonic signal processing method that excites a shear wave in a subject to analyze a propagation state of the shear wave by using a convex ultrasonic probe, the method comprising:
causing the ultrasonic probe to transmit a push wave for causing displacement in the subject;
causing the ultrasonic probe to transmit a detection wave after the transmission of the push wave, the detection wave passing through a region of interest which indicates an analysis target range in the subject;
receiving an ultrasonic wave reflected from the region of the interest by using the ultrasonic probe and converting the ultrasonic wave into a reception signal, the ultrasonic wave corresponding to the detection wave;
setting a plurality of observation points so that a distance between observation points along a propagation direction of the shear wave in the region of the interest is set to be not more than a distance between observation points along the propagation direction of the shear when a region closer to the ultrasonic probe than the region of the interest is set as a region of the interest and performing phasing addition for each of the plurality of the observation points to generate an acoustic line signal; and
calculating a mechanical property of the subject in the region of the interest based on the acoustic line signal for each of the plurality of the observation points.

12. A non-transitory recording medium storing a computer readable program causing a computer to execute ultrasonic signal processing that excites a shear wave in a subject to analyze a propagation state of the shear wave by using a convex ultrasonic probe, the ultrasonic signal processing comprising:
causing the ultrasonic probe to transmit a push wave for causing displacement in the subject;
causing the ultrasonic probe to transmit a detection wave following the transmission of the push wave, the detection wave passing through a region of interest which indicates an analysis target range in the subject;
receiving ultrasound reflected from the region of the interest by using the ultrasonic probe and converting the ultrasound into a reception signal, the ultrasound corresponding to the detection wave;
setting a plurality of observation points so that a distance between observation points along a propagation direction of the shear wave in the region of the interest is set to be not more than a distance between observation points along a propagation direction of a shear wave when a region closer to the ultrasonic probe than the region of the interest is set as the region of the interest and performing phasing addition for each of the plurality of the observation points to generate an acoustic line signal; and
calculating a mechanical property of the subject in the region of the interest based on the acoustic line signal for each of the plurality of the observation points.
